(19) United States
(12) Patent Application Publication
(10) Pub. No.: US 2025/0082700 A1
(43) Pub. Date: Mar. 13, 2025

(54) METHODS AND COMPOSITIONS OF TREATING BIOLFILM-RELATED DISEASES

(71) Applicant: UNIVERSITY OF ROCHESTER, Rochester, NY (US)

(72) Inventor: Jin XIAO, Rochester, NY (US)

(21) Appl. No.: 18/722,030

(22) PCT Filed: Feb. 16, 2023
(86) PCT No.: PCT/US23/13205

§ 371 Date: Jun. 20, 2024

Related U.S. Application Data
(60) Provisional application No. 63/268,420, filed on Feb. 23, 2022.

(51) Int. Cl. A61K 35/747; A61K 9/00; A61K 31/7048; A61K 35/00; A61K 38/16; A61P 1/02; A61P 31/04; A61P 31/10

(52) U.S. Cl. CPC A61K 35/747; A61K 9/0053; A61K 31/7048; A61K 38/164; A61P 1/02; A61P 31/04; A61P 31/10; A61K 2035/115

(57) ABSTRACT

A pharmaceutical composition for use in inhibiting formation of bacteria-based biofilms in a subject, said composition comprising 1) one or more Lactobacilli; and/or (2) a plantaricin, wherein the one or more Lactobacillus are selected from the group consisting of L. rhamnosus ATCC 2836, L. plantarum ATCC 8014, L. plantarum ATCC 14917, and L. salivarius ATCC 11741.

This application is a 371 national stage application of PCT International Application No. PCT/US23/13205, filed Feb. 16, 2023, which claims priority from U.S. Provisional Patent App. No. 63/268,420, filed Feb. 23, 2022, which is incorporated herein by reference.

This invention was made with government support under DE027412 and DE031025 awarded by the National Institutes of Health. The government has certain rights in the invention.

### FIELD

This application relates generally to the field of treatment of biofilm-related diseases.

### BACKGROUND

Dental caries, an ecological dysbiosis of oral microflora, initiates from the virulent biofilms formed on tooth surfaces where cariogenic bacteria and fungi metabolize dietary carbohydrates, produce acid, and lead to irreversible consequences—demineralization of tooth enamel. Streptococcus mutans is a well-known cariogenic pathogen due to its acidogenicity, aciduricity, and capability of synthesizing dental plaque extracellular matrix. Moreover, research also revealed the cariogenic role of oral Candida, in that it is: acidogenic, aciduric, capable of dissolving hydroxyapatite, and leads to more severe dental caries when infected together with Streptococcus mutans in the rat model. Children with oral Candida albicans presented with >5 times greater odds of experiencing early childhood caries (ECC) than children without this yeast strain. The presence of C. albicans in the oral cavity of preschool children was associated with oral bacterial dysbiosis with an abundance of taxa with greater virulence and more conducive for ECC. Furthermore, the emergence of S. mutans by one year was 3.5 times higher in infants with early colonization of oral Candida than those free of oral Candida. Therefore, regulating S. mutans and C. albicans simultaneously in the oral cavity shed new light on caries prevention.

### SUMMARY

One aspect of the present application relates to method for decolonizing or inhibiting formation of bacteria-based biofilms in a subject. The method comprises the step of administering to the subject an effective amount of (1) one or more Lactobacilli; and/or (2) a plantaricin, wherein the one or more Lactobacilli are selected from the group consisting of L. rhamnosus ATCC 2836, L. plantarum ATCC 8014, L. plantarum ATCC 14917, and L. salivarius ATCC 11741.

Another aspect of the present application relates to a method for preventing or treating a biofilm-related disease in a subject. The method comprises the step of administering to the subject an effective amount of (1) one or more Lactobacilli; and/or (2) a plantaricin, wherein the one or more Lactobacilli are selected from the group consisting of L. rhamnosus ATCC 2836, L. plantarum ATCC 8014, L. plantarum ATCC 14917, and L. salivarius ATCC 11741.

Another aspect of the present application relates to a method for preventing development of early childhood caries (ECC) in a newborn subject. The method comprise the step of administering to the mother of the newborn subject during pregnancy, an effective amount of a pharmaceutical composition comprising one or more Lactobacilli are selected from the group consisting of L. rhamnosus ATCC 2836, L. plantarum ATCC 8014, L. plantarum ATCC 14917, and L. salivarius ATCC 11741.

Another aspect of the present application relates to a pharmaceutical composition comprising one or more Lactobacilli are selected from the group consisting of L. rhamnosus ATCC 2836, L. plantarum ATCC 8014, L. plantarum ATCC 14917, and L. salivarius ATCC 11741; and a pharmaceutically acceptable carrier, wherein the pharmaceutical composition is formulated as a mouthwash, dental gel or dental coating.

### BRIEF DESCRIPTION OF THE DRAWINGS

FIG. 1 shows the growth curves of C. albicans, S. mutans and Lactobacilli in multispecies planktonic and biofilm conditions are plotted. The control group consists of C. albicans and S. mutans. The group with added Lactobacilli was marked as “with Lactobacillus”. (Panel A) Lactobacilli significantly inhibited the growth of C. albicans by 1 log after 6 h and 1-2 logs after 20 h incubation. (Panel B) Lactobacilli significantly inhibited the growth of S. mutans at 6 h and 20 h. S. mutans was inhibited to non-detectable level (<20 CFU/ml) after 20 h incubation with L. plantarum 8014 and L. salivarius 11741. (Panel C) Lactobacilli maintained a stable growth in all groups. (Panels D-L) The growth curves of C. albicans, S. mutans and Lactobacilli in multispecies biofilms condition are plotted. (Panels D-F) Lactobacilli (L. plantarum and L. salivarius) inhibited the growth of C. albicans in high sucrose condition (1% w/v) by 72 h, 3 log reduction comparing to the control group. No difference of C. albicans growth was detected with the addition of L. rhamnosus in all sugar conditions. (Panels G-I) Lactobacilli (L. plantarum and L. salivarius) inhibit the growth of S. mutans in high sugar condition (1% w/v sucrose and 1% w/v glucose). Significantly, L. plantarum 8014 and 14917 inhibited S. mutans in the biofilms to non-detectable level (<20 CFU/ml) as early as 48 h and the treated biofilms remained non-detectable S. mutans (<20 CFU/ml) at 72 h. L. rhamnosus had poor performance on inhibiting the growth of S. mutans growth in all sugar conditions. (Panels J-L) Lactobacilli maintained a stable growth in all groups. * Indicates the CFU values of the multispecies biofilms were significantly less than the control group at all follow-up time points (p<0.05). # Indicates the CFU values of the multispecies biofilms were significantly less than the control group at specific marked time point (p<0.05).

FIG. 2 shows quantitative measurement of microcolonies in 72 h multispecies biofilms (1% w/v sucrose condition). The 72 h biofilms of the control group (C. albicans and S. mutans) and experimental groups (with L. plantarum 14917) in 1% w/v sucrose condition were visualized by two-photon laser confocal microscope. The three-dimensional structure of the biofilms was rendered using Amira software. L. plantarum 14917 dramatically reduced biofilm formation, comparing to the control group. Biofilm dry weight was significantly reduced with added L. plantarum 14917 (panel A). * p<0.05. The biomass of the two biofilm components, bacteria and exopolysaccharides (EPS), was calculated using image-processing software COMSTAT (Heydorn et al., 2000). L. plantarum 14917 significantly reduced the biomass of bacteria and EPS (panel B). The confocal images indicate the cross-sectional and sagittal views of microcolonies formed in the control group (S. mutans and C. albicans duo-species) and with added L. plantarum 14917. Well-formed mushroom-shaped microcolonies were seen in the control group, and the largest size microcolonies were seen in the S. mutans and C. albicans duo-species biofilm. Microcolonies formed with added L. plantarum 14917 were much less structured. Bacteria components were less encapsulated with EPS. The amount of co-localization between bacteria and EPS was calculated using DUOSTAT (Panel E), which was consistent with the findings revealed in the images (* p<0.05). The surface-attached and free-floating microcolonies were evaluated using COMSTAT and DUOSTAT Software. Panel F illustrates biofilms treated by L. plantarum 14917 had significantly reduced microcolony size. (p>0.05; ANOVA, comparison for all pairs using Tukey-Kramer HSD).

FIG. 3 shows inhibitory effect of L. plantarum on clinically isolated C. albicans and S. mutans from children with early childhood caries in multispecies planktonic condition. The growth of C. albicans, S. mutans, and L. plantarum in multispecies planktonic conditions are plotted. C. albicans and S. mutans clinical strains were isolated from 10 children with early childhood caries (ECC). Experiments repeated in triplicates. Each planktonic multispecies condition included the C. albicans and S. mutans isolated from the same ECC child, with added L. plantarum 14917. The control group only consisted of C. albicans and S. mutans. (panel A) L. plantarum inhibited the growth of C. albicans by <1 log after 6 hours and approximately 2 logs at 20 hours (p<0.05 at 20 h). (panel B) L. plantarum significantly inhibited the growth of S. mutans at 6 hours by 1 log, and completely inhibited the growth of S. mutans after 20 hours (p<0.05 at 20 h). (panel C) L. plantarum maintained a stable growth during the 20 hours' interaction with S. mutans and C. albicans. (panel D) The culture medium pH dropped rapidly with the addition of L. plantarum in planktonic condition, and both groups reached the same acidic level at 20 hours.

FIG. 4 shows interaction of L. plantarum 14917 and clinically isolated C. albicans and S. mutans in multispecies biofilms. Multispecies biofilms were formed by L. plantarum 14917 and clinically isolated C. albicans, S. mutans from three children with ECC. The treated group was grown with added L. plantarum 14917. The growth of C. albicans, S. mutans, and L. plantarum 14917 in multispecies biofilms is plotted (panels A-C). In the treated group with L. plantarum 14917, C. albicans was reduced by 3 logs compared to the control group (panel A). At 48-h, after two times administration of L. plantarum 14917, S. mutans was inhibited entirely in biofilms of treatment group (panel B). L. plantarum dropped during the first 24 h due to the fact of low-sucrose (0.1%) condition and remained growing steady after 24-h within the high-sucrose (1%) condition (panel C). (panel D) The pH of the culture medium was significantly lower with added L. plantarum 14917 at 24, 48, and 72-h, compared to the control group (p<0.05). (panel E) The composition of each microorganism. L. plantarum 14917 became the dominant species after 48 hours of incubation. (panel F) Biofilm formation was significantly reduced with added L. plantarum 14917, with nearly 40% reduction of dry weight at 72 hours.

FIG. 5 shows changes of multispecies biofilm 3D structure by L. plantarum 14917. Biofilms were formed by C. albicans and S. mutans only (control) and treated by added L. plantarum 14917 in 1% sucrose condition and visualized by two-photon laser confocal microscope at 72 hours. L. plantarum 14917 dramatically reduced the biomass of bacteria (panel A) and EPS (panel B). Bacteria co-localized by EPS was significantly less in treatment group (panel C). Biofilm parameters are calculated using data from three biofilms formed by C. albicans and S. mutans isolated from three ECC children.

FIG. 6 shows inhibition of C. albicans hyphae formation by L. plantarum 14917. (panel A) S. mutans and C. albicans grown in 1% glucose at 48 hours. (panel B) S. mutans and C. albicans grown in 1% glucose with added L. plantarum 14917 at 48 hours. The addition of L. plantarum 14917 reduced the growth of C. albicans and inhibited the switching from yeast to hyphae and pseudohyphae form.

FIG. 7 shows regulation of S. mutans and C. albicans virulence genes by L. plantarum 14917 in multispecies biofilms. The expression of S. mutans genes related to carcinogenicity (gtfB, gtfC, and atpD) were reduced by approximately 50% in the 72 h-biofilms treated by L. plantarum 14917, comparing to the control group.

FIG. 8 shows a study design for determining effect of Lactobacillus species on clinically isolated C. albicans and S. mutans from children with early childhood caries.

FIG. 9 shows inhibitory effect of Lactobacillus species on clinically isolated C. albicans and S. mutans from children with early childhood caries in multispecies planktonic condition. The growth of C. albicans, S. mutans, and Lactobacillus spp. in multispecies planktonic condition are plotted. C. albicans and S. mutans clinical strains were isolated from two children with early childhood caries (ECC). Experiments repeated in triplicates. Each planktonic multispecies condition included the C. albicans and S. mutans isolated from the same ECC child, with added L. plantarum 14917. The control group only consisted of C. albicans and S. mutans. The treated group included Lactobacilli was marked as “with Lactobacillus spp.”. (panel A) All three Lactobacillus inhibited the growth of C. albicans by <1 log after 6 hours and 1-2 logs following 20 hours' incubation. (panel B) All three Lactobacillus inhibited the growth of S. mutans. The performance is ranked by L. plantarum 14917 and L. salivarius 11741, and L. plantarum 8014. All three Lactobacilli significantly inhibited the growth of S. mutans at 6 hours by 2 logs. L. plantarum 14917 and L. salivarius 11741 completely inhibited the growth of S. mutans after 20 hours, with the exception of L. plantarum 8014. (panel C) Lactobacilli maintained a stable growth during the 20 hours' interaction with S. mutans and C. albicans. (panel D) The culture medium pH dropped rapidly with the addition of Lactobacilli in planktonic condition, both control and treated groups reached the same acidic level at 20 hours.

FIG. 10 shows inhibition of C. albicans and S. mutans in ECC children by Lactobacilli in multispecies biofilms. Multispecies biofilms were formed by L. plantarum 14917 and L. salivarius 11741 and clinically isolated C. albicans, S. mutans from two children with ECC. The control group consists of C. albicans and S. mutans from the same ECC child. (panel A) L. plantarum and L. salivarius inhibited the growth of C. albicans by 3 logs compared to the control group. (panel B) At 48 hours, after two times administration of L. plantarum and L. salivarius, S. mutans was completely inhibited in biofilms of treatment group. (panel C) Lactobacilli maintained stable growth during 72-h incubation period with L. plantarum being significantly higher than L. salivarius. (panel D) The pH of the culture medium was significantly lower with added Lactobacilli at 24, 48 and 72 hours, comparing to the control group (p<0.05). (panels E-F) The composition of each microorganism is plotted. Lactobacilli became the dominate species after 48 h incubation.

FIG. 11 shows differential gene expression of S. mutans grown in treatment group vs. control group. Significant genes (>(−)1 Log2 fold change and FDR p value<0.05) that fit KEGG pathways are shown. Control group: S. mutans+C. albicans. Treatment group: L. plantarum 14917+S. mutans+C. albicans. All FDR p value that less than 1E-15 shown as 1E-15.

FIG. 12 shows differential gene expression of C. albicans grown in treatment group vs. control group. Significant genes (>(−)1 Log2 fold change and FDR p value<0.05) that fit KEGG pathways are shown. Control group: S. mutans+C. albicans. Treatment group: L. plantarum 14917+S. mutans+C. albicans. All FDR p value that less than 1E-15 shown as 1E-15.

FIG. 13 shows Differential gene expression of L. p 14917 grown in treatment group vs. single species biofilm. Significant genes (FDR p value<0.05) that fit KEGG pathways are shown. Treatment group: L. plantarum 14917+S. mutans+C. albicans. Single species biofilm: L. plantarum 14917. All FDR p value that less than 1E-15 shown as 1E-15.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” is also disclosed.

### I. Definitions

As used herein, the term “bacteria” refers to members of a large group of unicellular microorganisms that have cell walls but lack organelles and an organized nucleus.

As used herein, the term “Gram-positive bacteria” to bacteria characterized by having as part of their cell wall structure peptidoglycan as well as polysaccharides and/or teichoic acids and are characterized by their blue-violet color reaction in the Gram-staining procedure. Representative Gram-positive bacteria include: Actinomyces spp., Bacillus spp., Bifidobacterium spp., Clostridium spp., Clostridium spp., Corynebacterium spp., Enterococcus spp., Erysipelothrix spp., Eubacterium spp., Gardnerella spp., Gemella spp., Leuconostoc spp., Mycobacterium spp., Nocardia spp., Peptococcus spp., Peptostreptococcus spp., Proprionibacterium spp., Sarcina spp., Staphylococcus spp., and Streptococcus spp.

As used herein, the term “Gram-negative bacteria” refers to bacteria characterized by the presence of a double membrane surrounding each bacterial cell. Representative Gram-negative bacteria include Acinetobacter spp., Actinobacillus spp., Aggregatibacter spp., Aeromonas spp., Alcaligenes spp., Bacteroides spp., Bartonella spp., Bordetella spp., Borrelia spp., Branhamella spp., Brucella spp., Campylobacter spp., Chlamydia spp., Chromobacterium spp., Citrobacter spp., Eikenella spp., Enterobacter spp., Escherichia spp., Flavobacterium spp., Fusobacterium spp., Haemophilus spp., Helicobacter spp., Klebsiella pneumoniae, Klebsiella spp., Legionella spp., Leptospira spp., Moraxella spp., Morganella spp., Mycoplasma spp., Neisseria spp., Pasteurella spp., Plesiomonas spp., Prevotella spp., Proteus spp., Providencia spp., Pseudomonas spp., Rickettsia spp., Rochalimaea spp., Salmonella spp., Salmonella spp., Serratia spp., Shigella spp., Treponema spp., Veillonella spp., Vibrio spp., and Yersinia spp.

As used herein, the term “biofilm” refers to a sessile community of microorganisms characterized by cells that are attached to a substratum or interface or to each other, that are embedded in a matrix of extracellular polymers (more specifically extracellular polymers that they have produced), and that exhibit an altered phenotype with respect to growth rate and gene transcription (for example as, compared to their “non-biofilm”, free-floating or planktonic counterparts).

As used herein, the term “dental caries” refers to a biofilm-mediated, sugar-driven, multifactorial, dynamic disease that results in the phasic demineralization and remineralization of dental hard tissues. Caries can occur throughout life, both in primary and permanent dentitions, and can damage the tooth crown and, in later life, exposed root surfaces.

As used herein, the term “early childhood caries (ECC)”, formerly known as nursing bottle caries, baby bottle tooth decay, night bottle mouth and night bottle caries, is a disease that affects teeth in children aged between birth and 71 months. ECC is characterized by the presence of 1 or more decayed (noncavitated or cavitated lesions), missing (due to caries), or filled tooth surfaces in any primary tooth.

As used herein, the term “dental coating” is a material that is used in dentistry as a protective layer of the dental surface.

The phrase “pharmaceutically acceptable carrier or diluent” refers to any substance suitable for use in administering to an animal. In certain embodiments, a pharmaceutically acceptable carrier or diluent is sterile saline. In certain embodiments, such sterile saline is pharmaceutical grade saline.

As used herein, the term “subject” refers to a mammal, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). A “subject in need thereof” refers to a subject who may have, is diagnosed with, is suspected of having, or requires prevention of a biofilm-related disease or condition.

An “effective amount” or a “therapeutically effective amount” is defined herein in relation to the treatment or prevention of a biofilm-related disease or condition is an amount that when administered alone or in combination with another therapeutic agent to a cell, tissue, or subject is effective to decrease, reduce, inhibit, or otherwise abrogate the development of a biofilm-related disease or condition. An “effective amount” further refers to that amount of the compound sufficient to result in amelioration of symptoms, e.g., treatment, healing, prevention, or amelioration for the biofilm-related disease or condition, or in increase in the rate of treatment, healing, prevention, or amelioration of the biofilm-related disease or condition. When applied to an individual compound (active ingredient) administered alone, an “effective amount” refers to that ingredient alone. When applied to a combination, the “effective amount” refers to combined amounts of the active ingredients that result in the therapeutic effect, whether administered in combination, serially, or simultaneously. The “effective amount” will vary depending on the biofilm-related disease or condition and the severity of the biofilm-related disease or condition, as well as the age, weight, etc., of the subject to be treated. Additionally, the “effective amount” can vary depending upon the dosage form employed and the route of administration utilized. A physician or veterinarian having ordinary skill in the art can readily determine and prescribe the effective amount (e.g., ED50) of the active ingredients required. For example, the physician or veterinarian can start doses of the administered compounds at levels lower than that required in order to achieve the desired therapeutic effect and gradually increase the dosage until the desired effect is achieved.

### II. Methods of Inhibiting Formation of Microorganism-Based Biofilm and Treating Biofilm Related Diseases

One aspect of the present application is a method for preventing or treating a biofilm-related disease in a subject. The method comprises the step of administering to the subject an effective amount of a composition comprising (1) one or more Lactobacilli, and/or (2) plantaricin. The biofilm-related disease may be caused by bacteria and/or yeasts. In some embodiments, the biofilm-related disease is selected from the group consisting of dental caries, oral yeast infections, denture stomatitis, periodontitis, peri-implantitis, and burning mouth syndrome. In certain embodiments, the biofilm-related disease is early childhood caries (ECC).

In some embodiments, the biofilm-related disease is caused by a microorganism-based biofilm. The microorganism-based biofilms may be biofilms formed from any bacteria or yeast. In some embodiments, the microorganism-based biofilms comprise a bacterium selected from the group consisting of Streptococcus, Candida, Veillonella, Rothia, Actinomyces, Prevotella, Tannerella, Treponema, and Campylobacter, Lautropia. In some embodiments, the microorganism-based biofilms comprise Streptococcus mutans and/or Candida albicans.

In some embodiment, the composition comprises one or more Lactobacilli selected from the group consisting of L. rhamnosus ATCC 2836, L. plantarum ATCC 8014, L. plantarum ATCC 14917, and L. salivarius ATCC 11741.

In some embodiments, the composition is administered orally. In some embodiments, the composition is administered orally in the form of an orally consumable composition, such as a gum, candy or a beverage. In some embodiments, the composition is a pharmaceutical composition of the present application, such as a mouthwash, dental gel or dental coating.

Another aspect of the present application relates to a method for decolonizing or inhibiting formation of bacteria-based biofilms in a subject. The method comprises the step of administering to the subject an effective amount of a composition of the present application.

Another aspect of the present application is a method for preventing or treating dental caries in a subject. The method comprises the step of administering to the subject an effective amount of a composition comprising (1) one or more Lactobacilli selected from the group consisting of L. rhamnosus ATCC 2836, L. plantarum ATCC 8014, L. plantarum ATCC 14917, and L. salivarius ATCC 11741; and/or (2) plantaricin.

Another aspect of the present application is a method for inhibiting growth of Streptococcus mutans and/or Candida albicans in a subject. The method comprises the step of administering to the subject an effective amount of a pharmaceutical composition comprising (1) one or more Lactobacilli selected from the group consisting of L. rhamnosus ATCC 2836, L. plantarum ATCC 8014, L. plantarum ATCC 14917, and L. salivarius ATCC 11741 and/or (2) plantaricin. Another aspect of the present application is a method for inhibiting growth of Streptococcus mutans and/or Candida albicans in the oral cavity of a subject. The method comprises the step of administering to the subject an effective amount of a composition comprising one or more Lactobacillus selected from the group consisting of L. rhamnosus ATCC 2836, L. plantarum ATCC 8014, L. plantarum ATCC 14917, and L. salivarius ATCC 11741, wherein the composition is administered orally.

In some embodiments, the composition used in above-described methods comprises two, three or four Lactobacilli selected from the group consisting of L. rhamnosus ATCC 2836, L. plantarum ATCC 8014, L. plantarum ATCC 14917, and L. salivarius ATCC 11741. In some embodiments, the composition used in above-described methods further comprises an anti-fungal agent. In some embodiments, the anti-fungal agent is nystatin. In some embodiments, the composition used in above-described methods comprises L. plantarum 14917 and nystatin.

Another aspect of the present application is a method for preventing or treating dental caries in a subject. The method comprises the step of administering to the subject an effective amount of a pharmaceutical composition comprising an agent that inhibits the activity or expression of a bacterial gene. In some embodiments, the bacterial gene is selected from the group consisting of (1) the gtfB, gtfC and atpD genes of S. mutans and (2) the AAT22, ADE8, ALD5, AYR2, CAT1, CHA1, CHT2, ECM38, ERG4, FDH1, FOL1 GCV1, GCV2, HAL22, HPD1, IST1, LSC1, LSM6, MAL2, PCK1, PEX11, POX1-3, PXP2, SOD3, TEM1, THI20, THI6, URA3, HPW1, and ECE1 genes of C. albicans. In certain embodiments, the dental caries is early childhood caries (ECC). In some embodiments, the agent is selected from the group consisting of Lactobacillus and plantaricin. In some embodiments, the agent is selected from the group consisting of (1) Lactobacilli which is selected from the group consisting of L. rhamnosus ATCC 2836, L. plantarum ATCC 8014, L. plantarum ATCC 14917, and L. salivarius ATCC 11741, and (2) plantaricin.

Another aspect of the present application is a method for preventing or treating dental caries in a subject. The method comprises administering to the subject an effective amount of a pharmaceutical composition comprising one or more Lactobacilli selected from the group consisting of L. rhamnosus ATCC 2836, L. plantarum ATCC 8014, L. plantarum ATCC 14917, and L. salivarius ATCC 11741.

Another aspect of the present application relates to a method for preventing development of early childhood caries (ECC) in a newborn subject. The method comprises the step of administering to the mother of the newborn subject during pregnancy, an effective amount of a composition of the present application. In some embodiments, the composition comprises one or more Lactobacilli. In some embodiments, the one or more Lactobacilli are selected from the group consisting of L. rhamnosus ATCC 2836, L. plantarum ATCC 8014, L. plantarum ATCC 14917, and L. salivarius ATCC 11741.

In some embodiments, the composition or pharmaceutical composition described above is administered orally in the form of a mouthwash, dental gel or dental coating. In some embodiments, the mouthwash, dental gel or dental coating comprises one or more Lactobacilli at an individual or total dose of 10⁷-10⁸ CFU/ml, 10⁷-10⁹ CFU/ml, 10⁷-10¹⁰ CFU/ml, 10⁷-10¹¹ CFU/ml, 10⁷-10¹² CFU/ml, 10⁸-10⁹ CFU/ml, 10⁸-10¹⁰ CFU/ml, 10⁸-10¹¹ CFU/ml, 10⁸-10¹² CFU/ml, 10⁹-10¹⁰ CFU/ml, 10⁹-10¹⁰ CFU/ml, 10⁹-10¹² CFU/ml, 10¹⁰-10¹¹ CFU/ml, 10¹⁰-10¹² CFU/ml, or 10¹¹-10¹² CFU/ml.

In some embodiments, the mouthwash, dental gel or dental coating comprises one or more Lactobacilli selected from the group consisting of L. rhamnosus ATCC 2836, L. plantarum ATCC 8014, L. plantarum ATCC 14917, and L. salivarius ATCC 11741 at an individual or total dose of 10⁷-10⁸ CFU/ml, 10⁷-10⁹ CFU/ml, 10⁷-10¹⁰ CFU/ml, 10⁷-10¹¹ CFU/ml, 10⁷-10¹² CFU/ml, 10⁸-10⁹ CFU/ml, 10⁸-10¹⁰ CFU/ml, 10⁸-10¹¹ CFU/ml, 10⁸-10¹² CFU/ml, 10⁹-10¹⁰ CFU/ml, 10⁹-10¹¹ CFU/ml, 10⁹-10¹² CFU/ml, 10¹⁰-10¹¹ CFU/ml, 10¹⁰-10¹² CFU/ml, or 10¹¹-10¹² CFU/ml.

In some embodiments, the mouthwash, dental gel or dental coating comprises L. plantarum 14917 at a dose of 10⁷-10⁸ CFU/ml, 10⁷-10⁹ CFU/ml, 10⁷-10¹⁰ CFU/ml, 10⁷-10¹¹ CFU/ml, 10⁷-10¹² CFU/ml, 10⁸-10⁹ CFU/ml, 10⁸-10¹⁰ CFU/ml, 10⁸-10¹¹ CFU/ml, 10⁸-10¹² CFU/ml, 10⁹-10¹⁰ CFU/ml, 10⁹-10¹¹ CFU/ml, 10⁹-10¹² CFU/ml, 10¹⁰-10¹¹ CFU/ml, 10¹⁰-10¹² CFU/ml, or 10¹¹-10¹² CFU/ml.

In some embodiments, the mouthwash, dental gel or dental coating comprises two Lactobacilli selected from the group consisting of L. rhamnosus ATCC 2836, L. plantarum ATCC 8014, L. plantarum ATCC 14917, and L. salivarius ATCC 11741, wherein the two Lactobacilli are present in the mouthwash, dental gel or dental coating at a CFU ratio in the range of 1:10 to 10:1 or 1:5 to 5:1, or 1:3 to 3:1. In one embodiments, the mouthwash, dental gel or dental coating comprises L. plantarum ATCC 14917 and L. plantarum ATCC 8014 at a CFU ratio of 1:1 (e.g., 10⁸ CFU/ml of L. plantarum ATCC 14917 and 10⁸ CFU/ml of L. plantarum ATCC 8014).

In some embodiments, the mouthwash, dental gel or dental coating comprises plantaricin at a concentration in the range of 20-40 ng/ml, 20-100 ng/ml, 20-200 ng/ml, 20-400 ng/ml, 20-1000 ng/ml, 20-2000 ng/ml, 40-100 ng/ml, 40-200 ng/ml, 40-400 ng/ml, 40-1000 ng/ml, 40-2000 ng/ml, 100-200 ng/ml, 100-400 ng/ml, 100-1000 ng/ml, 100-2000 ng/ml, 200-400 ng/ml, 200-1000 ng/ml, 200-2000 ng/ml, 400-1000 ng/ml, 400-2000 ng/ml, or 1000-2000 ng/ml. In some embodiments, the mouthwash, dental gel or dental coating comprises plantaricin at a concentration in the range of 200-400 ng/ml.

In some embodiments, the mouthwash, dental gel or dental coating is applied once per day, twice per day or three times a day for a period of 1-60, 1-45, 1-30, 1-15, 1-10, 1-5 or 1-3 days. In some embodiments, the mouthwash, dental gel or dental coating is applied once per day, twice per day or three times a day for a period of at least 1, 2, 3, 4, 5, 6, 7 or 8 weeks.

In some embodiments, the composition is administered orally in the form of a mouthwash, dental gel or dental coating. In some embodiments, the mouthwash, dental gel or dental coating is applied once per day, twice per day or three times a day for a period of 1-60, 1-45, 1-30, 1-15, 1-10, 1-5 or 1-3 days. In some embodiments, the mouthwash, dental gel or dental coating is applied once per day, twice per day or three times a day for a period of at least 1, 2, 3, 4, 5, 6, 7 or 8 weeks.

One of ordinary skill will understand that the composition used in the methods described herein may be delivered in forms including, but not limited to, an oral formulation, capsule formulation, tablet formulation, infusion, etc. One of ordinary skill will understand that the particular formulation or method of delivery of the composition is not limiting on the methods described herein.

### III. Compositions of the Present Application

Another aspect of the present application relates to compositions that can be used for preventing or treating a biofilm-related disease. The composition comprises (1) one or more Lactobacilli and/or (2) plantaricin. In some embodiments, the one or more Lactobacilli is selected from the group consisting of L. rhamnosus ATCC 2836, L. plantarum ATCC 8014, L. plantarum ATCC 14917, and L. salivarius ATCC 11741. In some embodiments, the composition comprises (1) one or more Lactobacilli selected from the group consisting of L. rhamnosus ATCC 2836, L. plantarum ATCC 8014, L. plantarum ATCC 14917, and L. salivarius ATCC 11741, and (2) plantaricin. In some embodiments, the composition comprises two or more Lactobacilli selected from the group consisting of L. rhamnosus ATCC 2836, L. plantarum ATCC 8014, L. plantarum ATCC 14917, and L. salivarius ATCC 11741. In some embodiments, the composition comprises (1) two or more Lactobacilli selected from the group consisting of L. rhamnosus ATCC 2836, L. plantarum ATCC 8014, L. plantarum ATCC 14917, and L. salivarius ATCC 11741, and (2) plantaricin.

In some embodiments, the composition further comprises an antifungal agent. In some embodiments, the antifungal agent is nystatin.

In some embodiments, the composition of the present application is in the form of an orally consumable product. The term “orally consumable product” refers to a composition that can be drunk, eaten, swallowed, ingested or otherwise in contact with the mouth of man or animal. Orally consumable products are safe for human or animal consumption when used in a generally acceptable range. Examples of orally consumable products include, but are not limited to, candies, gums, beverages, and dairy products such as yogurts.

In some embodiments, the composition of the present application is formulated as pharmaceutical composition that comprises a pharmaceutically acceptable carrier. In some embodiments, the pharmaceutical composition is formulated for oral administration. In some embodiments, the pharmaceutical composition is formulated as a mouthwash, dental gel or dental coating.

In some embodiments, the composition of the present application is formulated as a liquid or hydrogel formulation. In some embodiments, the liquid or hydrogel formulation has a pH value in the range of 3-9, 3-8, 3-7, 3-6, 3-5, 3-4, 4-9, 4-8, 4-7, 4-6, 4-5, 5-9, 5-8, 5-7, 5-6, 6-9, 6-8, 6-7, 7-9, 7-8, or 8-9. In some embodiments, the liquid or hydrogel formulation has a pH value in the range of 4-7. In some embodiments, the liquid or hydrogel formulation is in the form of a mouthwash, dental gel or dental coating.

In some embodiments, the composition of the present application is formulated as a liquid or hydrogel formulation comprising 0.1-0.3%, 0.1-0.6, 0.1-1%, 0.1-3%. 0.1-6%, 0.1-10%, 0.3-0.6, 0.3-1%, 0.3-3%. 0.3-6%, 0.3-10%, 0.6-1%, 0.6-3%. 0.6-6%, 0.6-10%, 1-3%. 1-6%, 1-10%, 3-6%, 3-10%, 6-10% (w/w or w/v) sugar. Examples of sugar include, but are not limited to sucrose, glucose, galactose, fructose and galacto oligosaccharide. In some embodiments, the liquid or hydrogel formulation composition comprises about 1% (w/w or w/v) sugar. In some embodiments, the liquid or hydrogel formulation composition comprises (1) L. plantarum, and (2) 1% (w/w or w/v) sucrose, or 1% (w/w or w/v) glucose, or 1% (w/w or w/v) galacto oligosaccharide.

In some embodiments, the composition of the present application is formulated as the pharmaceutical composition comprises one or more carriers suitable for delivering the therapeutic agents (e.g., Lactobacilli, plantaricin and/or antifungal agents) to a target tissue/organ, such as tooth or gum tissue. Exemplary carriers for delivery include solutions, hydrogels, nanoparticles, lipids, liposomes, micelles, polymers, polymeric micelles, emulsions, polyelectrolyte complexes, microcapsules and combinations thereof, and pegylated derivatives thereof.

Exemplary nanoparticles include paramagnetic nanoparticles, superparamagnetic nanoparticles, metal nanoparticles, polymeric nanoparticles, nanoworms, nanoemulsions, nanogels, fullerene-like materials, inorganic nanotubes, dendrimers (such as with covalently attached metal chelates), nanocapsules, nanospheres, nanofibers, nanohoms, nano-onions, nanorods, nanoropes and quantum dots.

In other embodiments, a polymeric nanoparticle is made from a synthetic biodegradable polymer, a natural biodegradable polymer or a combination thereof. Synthetic biodegradable polymers can include, polyesters, such as poly(lactic-co-glycolic acid)(PLGA) and polycaprolactone; polyorthoesters, polyanhydrides, polydioxanones, poly-alkyl-cyano-acrylates (PAC), polyoxalates, polyiminocarbonates, polyurethanes, polyphosphazenes, or a combination thereof. Natural biodegradable polymers can include starch, hyaluronic acid, heparin, gelatin, albumin, chitosan, dextran, or a combination thereof.

In certain embodiments, pharmaceutical compositions provided herein include one or more the therapeutic agents (e.g., Lactobacilli, plantaricin and/or antifungal agents) and one or more excipients. Exemplary excipients include water, salt solutions, alcohol, polyethylene glycols, gelatin, lactose, amylase, magnesium stearate, talc, silicic acid, viscous paraffin, hydroxymethylcellulose, polyvinylpyrrolidone and combinations thereof.

In some embodiments, the pharmaceutical composition comprises a buffering agent to maintain a desired pH range. In some embodiments, the buffering agent is a buffering agent for oral use. Examples of buffering agents for oral use include, but are not limited to, phosphate-buffered saline (PBS), potassium chloride, sodium chloride, magnesium chloride, calcium chloride, potassium thiocyanate, and sodium bicarbonate.

The pharmaceutical composition of the present application is formulated in accordance with the particular route of administration. In some embodiments, the pharmaceutical composition of the present application is formulated for oral administration. In some embodiments, pharmaceutical composition of the present application is formulated as a mouthwash, dental gel or dental coating.

The present application is further illustrated by the following examples that should not be construed as limiting. The contents of all references, patents, and published patent applications cited throughout this application, as well as the Figures and Tables, are incorporated herein by reference.

### EXAMPLES

### Example 1. Materials and Methods

### Bacterial Strains and Starter Preparation

The microorganisms used in the study were S. mutans UA159, C. albicans SC5314, L. rhamnosus ATCC 2836, L. plantarum ATCC 8014, L. plantarum ATCC 14917 and L. salivarius ATCC 11741. C. albicans, S. mutans and Lactobacillus were recovered from frozen stock using YPD agar (BD Difco™, 242720), Blood agar (TSA with Sheep Blood, Thermo Scientific™ R01202) and MRS agar (BD Difco™, 288210) respectively. After 48 h incubation, 3-5 colonies of each species were inoculated into 10 ml of broth for overnight incubation (5% CO₂, 37° C.). C. albicans was grown in YPD broth (BD Difco™, 242820); S. mutans was grown in TSBYE broth (3% Tryptic Soy, 0.5% Yeast Extract Broth, BD Bacto™ 286220 and Gibco™ 212750) with 1% glucose; and Lactobacillus spp. were grown in MRS broth (BD Difco™ 288130). On the following day, 0.5 ml of the overnight starters were added to individual glass tubes with fresh broth and incubated for 3-4 h to reach mid-exponential phase with desirable optical density. The morning starters were then ready for the preparation of planktonic and biofilm models described below.

### Planktonic Model

Interactions between C. albicans, S. mutans and Lactobacillus species were first evaluated in planktonic conditions. The inoculation quantity of C. albicans (10³ CFU/ml) and S. mutans (10⁵ CFU/ml) was chosen to simulate high caries risk conditions in the clinical setting. The inoculation quantity of the four Lactobacillus (10⁸ CFU/ml) is the lower dose of the probiotics used in the commercial probiotic products (10⁹-10¹² CFU as a single dosage). C. albicans, S. mutans and one of the Lactobacilli were grown in 10 ml TSBYE broth with 1% glucose for 20 h (5% CO₂, 37° C.). Additionally, a dose-titration effect of L. plantarum 14917 (10⁴-10⁷ CFU/ml inoculation) was assessed. The growth of each microorganism and pH values were measured at multiple time points.

### Mixed-Species Biofilm Model

The study then used a mixed-species biofilm model to assess the effect of Lactobacilli on the biofilm formation by S. mutans and C. albicans. The biofilm was formed on saliva coated hydroxyapatite discs (0.50″ diameter×0.05″ thickness, Clarkson Chromatography Products, Inc., South Williamsport, PA). The discs were placed in a vertical position using a custom-made disc holder to mimic the caries-prone smooth tooth surfaces in the oral cavity (Xiao, J., et al. (2012). PLoS Pathog 8(4), e1002623).

The mixture of S. mutans, C. albicans and Lactobacilli was inoculated in 2.8 ml of TSBYE broth with 0.1% (w/v) sucrose, and incubated at 37° C. and 5% CO₂. During the first 24 h, the organisms were grown undisturbed to allow initial biofilm formation. At 24 h, the biofilms were transferred to a fresh culture medium containing 1% (w/v) sucrose or 1% (w/v) glucose to induce cariogenic challenges, while an additional set of biofilms was grown with 0.1% (w/v) sucrose. The culture medium was replaced every 24 h until the end of the experimental period (72 h). Lactobacilli (10⁸ CFU/ml) was added to the fresh culture medium daily. The culture medium pH was measured at selected time points. The biofilms underwent microbiological, dry-weight, and confocal imaging assays at 24, 48, and 72 h, transcriptome analysis via RNA-Seq at 48 h, and qRT-PCR validation at 48, 50, and 52 h. Methods detailed in Xiao, et al. (2012) Supra. Duplicated discs were used in each run. Independent assays were repeated three times.

Inhibition of C. albicans and S. mutans by L. plantarum Supernatant

The supernatant of L. plantarum 8014 and 14917 overnight culture was harvested and sterilized with a vacuum filter system (0.22 μm PES, Corning™ Disposable Vacuum Filter Systems, USA). S. mutans and C. albicans with a range of concentration (10¹⁻⁸ for S. mutans and 10¹⁻⁶ for C. albicans) were treated with the supernatant of L. plantarum and allowed to grow for 24 h in TSBYE with 1% (w/v) glucose or 1% (w/v) sucrose condition in 96-well plates. Clear culture indicated no growth of microorganisms.

Inhibition of S. mutans and C. albicans by Plantaricin

Bacteriocins, antimicrobial molecules, produced by L. plantarum are known as plantaricins. Peptide plantaricin-149 (acetate) powers (Creative Peptides, Shirley, USA) were dissolved in ddH₂O to prepare plantaricin solutions. S. mutans (3.6×10³ CFU/ml) and C. albicans (3.1×10¹ CFU/ml) from 1 representative S-ECC child were selected and treated with the plantaricin with a range of concentration (0-400 μg/ml). The mixtures of plantaricin with S. mutans or C. albicans were grown for 24 hours in TSBYE with 1% glucose in 96-well plates. Clear culture after 24 hours' incubation indicated no growth of microorganisms. Therefore, the minimal inhibition concentration (MIC) of plantaricin-149 was defined as the lowest concentration that inhibited the growth of S. mutans and C. albicans.

### Transcriptome Analysis by RNA-Seq—RNA Library Preparation and Sequencing

The mass of biofilms was harvested from four discs for each condition. The discs were immersed in RNALater (Applied Biosystems/Ambion, Austin, TX, United States) for 1 hour, followed by biomass removal with a spatula. RNAs were extracted and purified with MasterPure complete DNA and RNA purification kit (epicenter, Lucigen, Widconsin, United States). Raw RNA product was quantified using NanoDrop One Microvolume UV-Vis Spectrophotometer (Thermo Scientific™, Wilmington, DE, United States). rRNA depletion was performed using Ribozero rRNA Removal Kit (Illumina, San Diego, CA, USA). RNA sequencing library was prepared using NEBNext Ultra RNA Library Prep Kit for Illumina by following the manufacturer's recommendations (NEB, Ipswich, MA, USA). The sequencing libraries were multiplexed and clustered on one lane of a flow cell and loaded on the Illumina HiSeq instrument according to manufacturer's instructions.

RNA sequencing library was prepared using NEBNext Ultra RNA Library Prep Kit for Illumina by following the manufacturer's recommendations (NEB, Ipswich, MA, USA). Briefly, enriched RNAs were fragmented for 15 minutes at 94° C. First and second strand cDNA were synthesized. The cDNA fragments were end repaired and adenylated at 3′ends, and universal adapter was ligated to cDNA fragments, followed by index addition and library enrichment with limited cycle PCR. Sequencing libraries were validated using the Agilent Tapestation 4200 (Agilent Technologies, Palo Alto, CA, USA), and quantified by using Qubit 2.0 Fluorometer (Invitrogen, Carlsbad, CA) as well as by quantitative PCR (Applied Biosystems, Carlsbad, CA, USA).

The sequencing libraries were multiplexed and clustered on one lane of a flow cell and loaded on the Illumina HiSeq instrument according to manufacturer's instructions. The samples were sequenced using a 2×150 Paired End (PE) configuration. Image analysis and base calling were conducted using the HiSeq Control Software (HCS). Raw sequence data generated from Illumina HiSeq was converted into FASTQ files and de-multiplexed using Illumina's bcl2fastq 2.17 software. One mis-match was allowed for index sequence identification. After demultiplexing, sequence data was checked for overall quality and yield. The sequence reads were trimmed to remove possible adapter sequences and nucleotides with poor quality using Trimmomatic v.0.36. The STAR aligner v.2.5.2b (Dobin, A., et al. (2013). Bioinformatics 29(1), 15-21) was used to map the trimmed reads to the reference genomes. Unique gene hit counts were calculated by using feature Counts from the Subread package v.1.5.2. Only unique reads within exon regions were counted. Gene hit counts were extracted and the gene hit counts table was used for downstream differential expression analysis.

Using DESeq2, a comparison of gene expression between the groups of samples was performed. The Wald test was used to generate p-values and Log2 fold changes. S. mutans and C. albicans genes with adjusted p-values (False Discovery Rate (FDR) p-values)<0.05 and absolute log 2 fold changes>2 and L. plantarum 14917 genes with FDR p-values<0.05 and absolute log 2 fold changes>1 were called as differentially expressed genes (DEGs) for each comparison. A gene ontology (GO) analysis was performed on the statistically significant set of genes by implementing the software GeneSCF v1.1 (Subhash, S., et al. (2016). Bmc Bioinformatics 17). The GO list was used to cluster the set of genes based on their biological process and determine their statistical significance. A Principal component analysis (PCA) was performed using the “plotPCA” function within the DESeq2 R package. The plot shows the samples in a 2D plane spanned by their first two principal components. The top 500 genes, selected by highest row variance, were used to generate the plot. Volcano plots were created by VolcaNoseR (Goedhart, J., et al. (2020). Scientific Reports 10(1)). Kyoto Encyclopedia of Genes and Genomes pathways were generated by KEGG mapper (genome.jp) and Cytoscape software version 3.8.2.

Real-Time Reverse Transcription Polymerase Chain Reaction (qRT-PCR)

Then cDNAs were synthesized using 0.2 g of purified RNA and the BioRad iScript cDNA synthesis kit (Bio-Rad Laboratories, Inc., Hercules, CA). The resulting cDNA and negative controls were amplified by quantitative amplification condition using Applied Biosystems™ PowerTrack™ SYBR Green Master Mix and a QuantStudio™ 3 Real-Time PCR System (Thermo Fisher Scientific, United States). Each 20 μL reaction mixture included template cDNA, 10 μM each primer, and 2× SYBR-Green mix (containing SYBR-Green and Taq DNA Polymerase). Unique core genes of S. mutans, C. albicans and L. plantarum were used as internal reference for comparative expression calculation: gyrA for S. mutans genes (Zeng, L., et al. (2013). J Bacteriol 195(4), 833-843); ACT1 for C. albicans, and ropB for L. plantarum.

### Laser Scanning Confocal Fluorescence Microscopy (LCSFM) Imaging of Biofilm Matrix

The study assessed two essential components of biofilm matrix: bacteria and [0078] exopolysaccharides (EPS) using LCSFM, (Xiao et al., 2012) Supra. Briefly, 1 μM Alexa Fluor® 647-labeled dextran conjugate (Molecular Probes, Invitrogen Corp., Carlsbad, CA) was added to the culture medium from the beginning of and during the development of the biofilms for exopolysaccharides visualization. The bacterial species and fungal species were labeled by SYTO® 9 green fluorescent nucleic acid stain (485/498 nm; Molecular Probes). The images were obtained using an Olympus FV 1000 two photon laser scanning microscope (Olympus, Tokyo, Japan) equipped with a 10× (0.45 numerical aperture) water immersion objective lens. Each biofilm formed on the HA disc was scanned at 5 positions randomly (Xiao, J., et al. (2010). J Appl Microbiol 108(6), 2103-2113). Three independent biofilm experiments were performed, and 10 image stacks were collected for each experiment. Amira 5.0.2 (Mercury Computer Systems Inc., Chelmsford, MS) was used to create 3D renderings of EPS and bacteria of the biofilms detailed previously (Klein, M. I. et al. (2011). J Vis Exp (47)). COMSTAT and DUOSTAT (http://www.imageanalysis.dk) were used for biofilm quantitative analysis, including biomass, number and size (volume, diameter, and height) of microcolonies, and the co-localization of EPS and bacteria across the biofilms (Xiao et al., 2012) Supra.

### Microbiological Analysis of the Mixed-Species Bacterial Population

The biofilms were homogenized by sonication. The homogenized suspension was used to determine the number of viable cells by plating on blood agar using an automated EddyJet Spiral Plater (IUL, SA, Barcelona, Spain). Three species were differentiated by colony morphology in conjunction with microscopic examination of cells from selected colonies (Guggenheim, B., et al. (2001). (J Dent Res 80(1), 363-370).

### Statistical Analysis

To compare the abundance of S. mutans, C. albicans and Lactobacillus spp. in planktonic and biofilm conditions, the CFU values were first converted to natural log values, zero values remained to be zero. The log values were compared between each group treated with Lactobacillus spp to the control group using Mann-Whitney U test after assessing the normality of data. For other measurements, such as biomass (bacteria and EPS), number and size of microcolonies, and pH value of the biofilms at specific time points, normality tests were performed first. For normally distributed data, the comparisons between groups were tested using t-test for two groups and one-way ANOVA for more than two groups followed by post hoc test. For data that were not normally distributed, Kruskal-Wallis was used to compare the outcomes of more than two groups, and Mann-Whitney U test was used for two groups comparison. Statistical tests were two-sided with a significant level of 5%. IBM SPSS was used for statistical analyses.

### Example 2. Macrophages Inhibition of C. albicans and S. mutans by Lactobacilli in Planktonic Condition

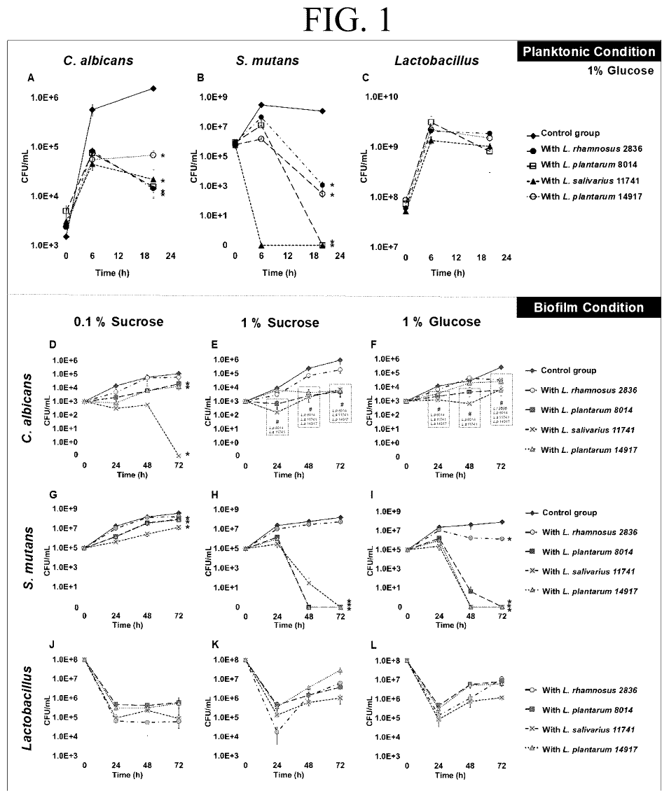

All four Lactobacillus spp. significantly inhibited the growth of C. albicans by 1 log at 6 h and 2 logs at 20 h (FIG. 1, panel A) in planktonic conditions. All tested Lactobacilli significantly inhibited the growth of S. mutans at 6 h and 20 h (FIG. 1, panel B). In contrast to the inhibited growth of C. albicans and S. mutans, the growth of Lactobacilli in multispecies-condition was not different from their growth in a single Lactobacillus species condition (FIG. 1, panel C). The culture medium pH dropped faster with the addition of Lactobacilli spp, however, reached the same acidity (˜4) at 20 h across all conditions (p>0.05). A dose-dependent effect was seen, the minimal inoculum of L. plantarum 14917 that demonstrated inhibition on the growth of S. mutans and C. albicans was 10⁸ CFU/ml.

### Example 3. Inhibition of C. albicans and S. mutans by Lactobacillus in Multispecies Biofilms

The growth of C. albicans and S. mutans were significantly inhibited by L. salivarius 11741, L. plantarum 8014, and L. plantarum 14917 in multispecies biofilms (FIG. 1D-L). Interestingly, rich sucrose condition (1% sucrose vs. 0.1% sucrose) enhanced the performance of Lactobacillus spp. Intriguingly, L. plantarum 8014 and 14917 inhibited S. mutans to non-detectable levels (<20 CFU/ml) as early as 48 h and the inhibitory effect remained until 72 h. Whereas, L. rhamnosus did not inhibit the growth of S. mutans growth except in 1% (w/v) glucose condition. The dynamic changes of microorganism composition in each condition were plotted. In the biofilms treated with L. salivarius 11741, L. plantarum 8014 and L. plantarum 14917 (1% (w/v) sucrose and 1% (w/v) glucose conditions), Lactobacilli became the dominate species after 48 h. The pH of the culture medium was significantly lower with added Lactobacilli at 24, 48 and 72 h, comparing to the control group (p<0.05).

### Example 4. Inhibition of Cariogenic Biofilm Formation by L. plantarum

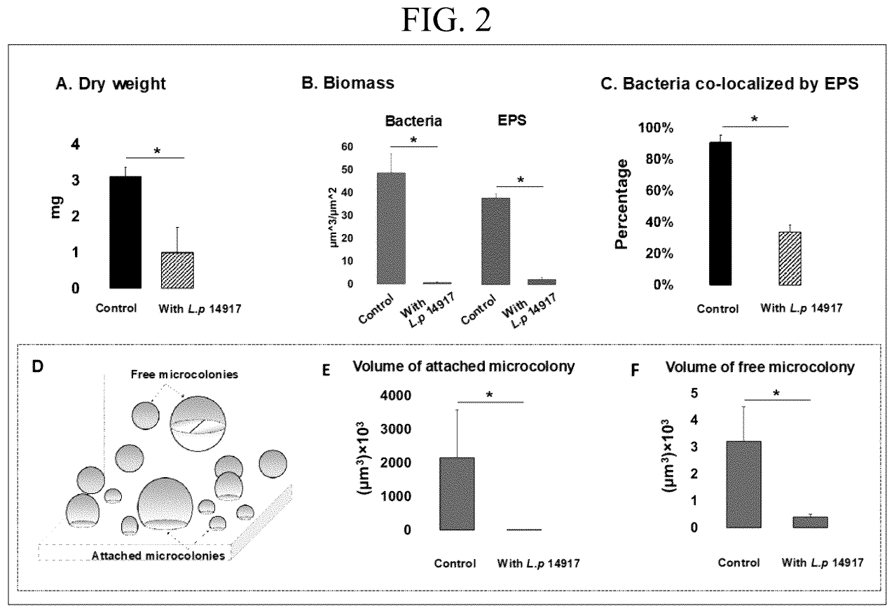

Since L. plantarum 8014 and 14917 demonstrated the better inhibition of C. albicans and S. mutans in planktonic and biofilm conditions, these two strains advanced to the biofilm structural analysis. L. plantarum 8014 and 14917 significantly reduced cariogenic biofilm formation measured by bacteria and EPS biomass and biofilm dry-weight (p<0.05), comparing to the control group (C. albicans-S. mutans duo-species biofilm). The 72 h biofilms are shown in FIG. 2, panel A, the dynamic changes of biofilm formation from 24-72 h are shown in FIG. 2, panel B. The vertical distributions of bacteria and EPS further demonstrate the altered biofilm assembly (FIG. 2, panel C). The control group formed the thickest biofilms in 1% sucrose condition, with the bulk of the biofilm accumulated at around 150-250 μm above the biofilm-HA disc interface. Conversely, the biofilms treated by L. plantarum 14917 were the thinnest and had the least horizontal converge, with approximately 15% coverage of bacteria and 19% EPS at the most abundant layer (20 μm above the biofilm-HA disc interface).

Microcolonies are considered virulent and functional structures of biofilm. Surface-attached and free-floating microcolonies were identified in the biofilms. Well-formed mushroom-shaped microcolonies formed in the control group (FIG. 2, panel A). Microcolonies formed with added L. plantarum 14917 were less structured, with less bacteria components enmeshed with EPS (FIG. 2, panel E) (p<0.05). Furthermore, biofilms treated by L. plantarum 14917 had significantly fewer surface-attached and free-floating microcolonies, with reduced size (FIG. 2, panel F).

### Example 5. Inhibition on C. albicans and S. mutans Growth by L. plantarum Supernatant

The supernatant of L. plantarum 14917 demonstrated antibacterial and antifungal activity against C. albicans and S. mutans. Specifically, the supernatant of L. plantarum 14917 inhibited the growth of S. mutans with a starting concentration equal or lower than 10⁴ CFU/ml in 1% sucrose condition, and the growth of C. albicans with a starting concentration equal or lower than 10¹ CFU/ml in 1% sucrose condition. The supernatant of L. plantarum 8014 had no inhibitory effect on C. albicans. The inhibitory effect was identified as bacteriostatic and fungistatic.

### Example 6. Transcriptomic Analysis

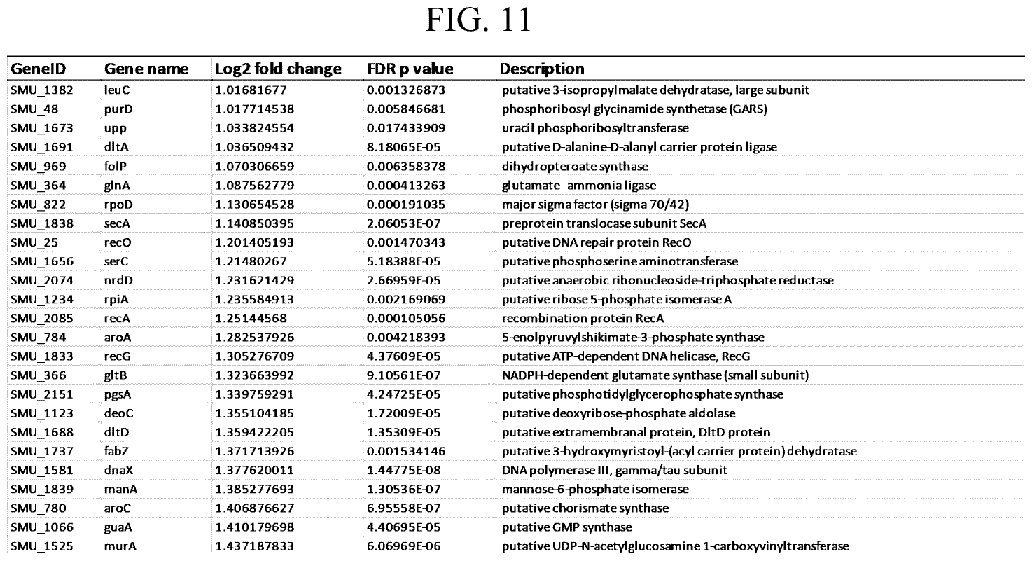

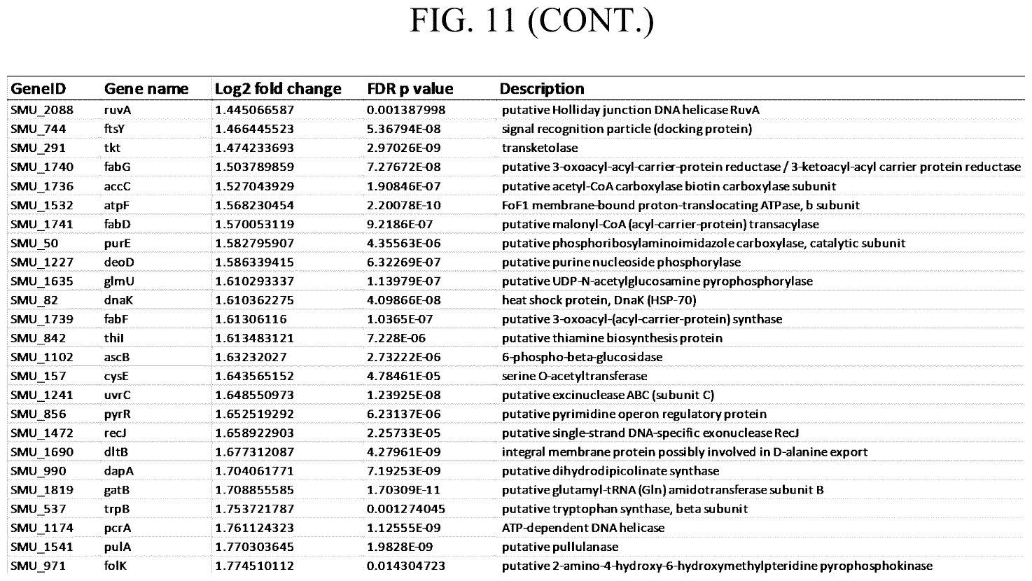

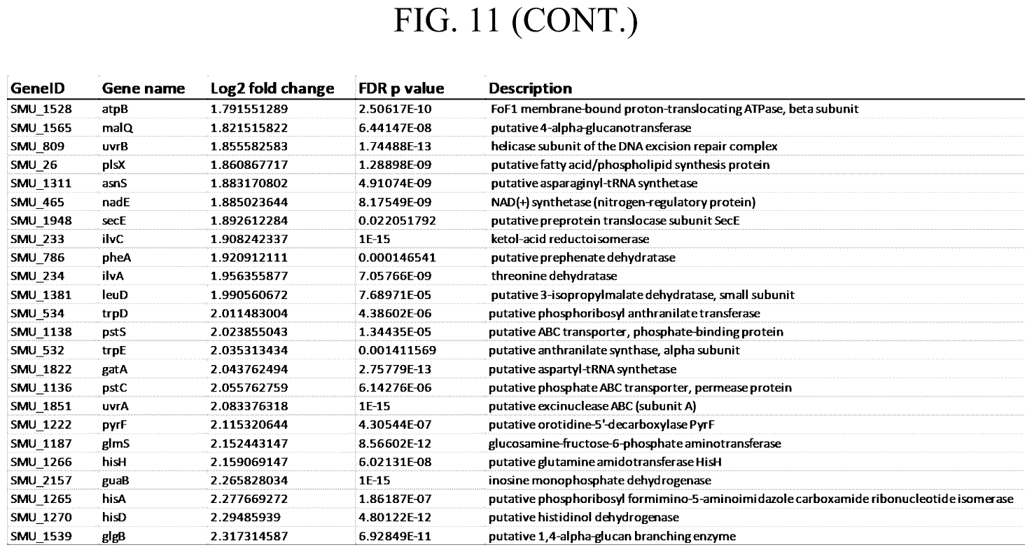

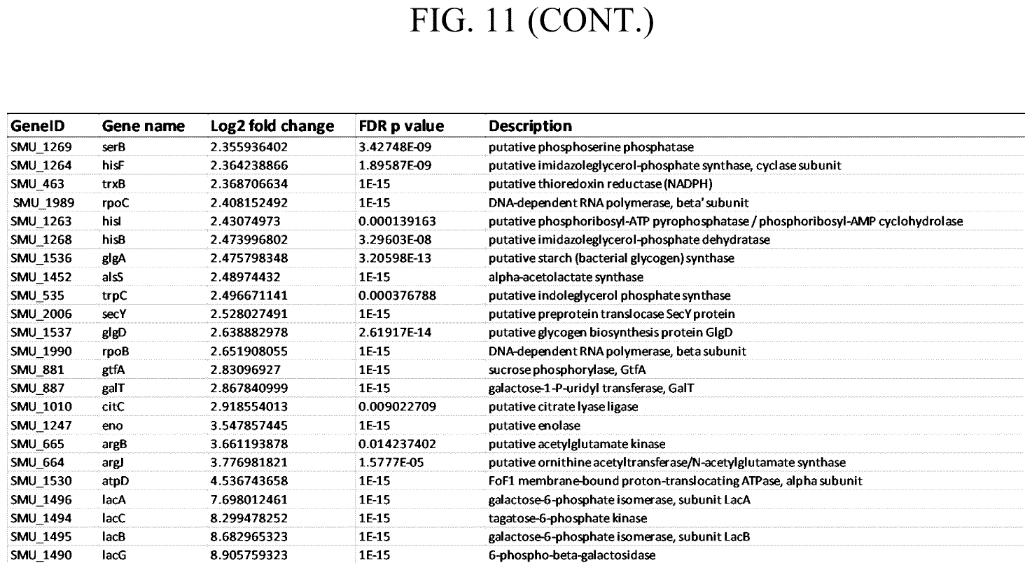

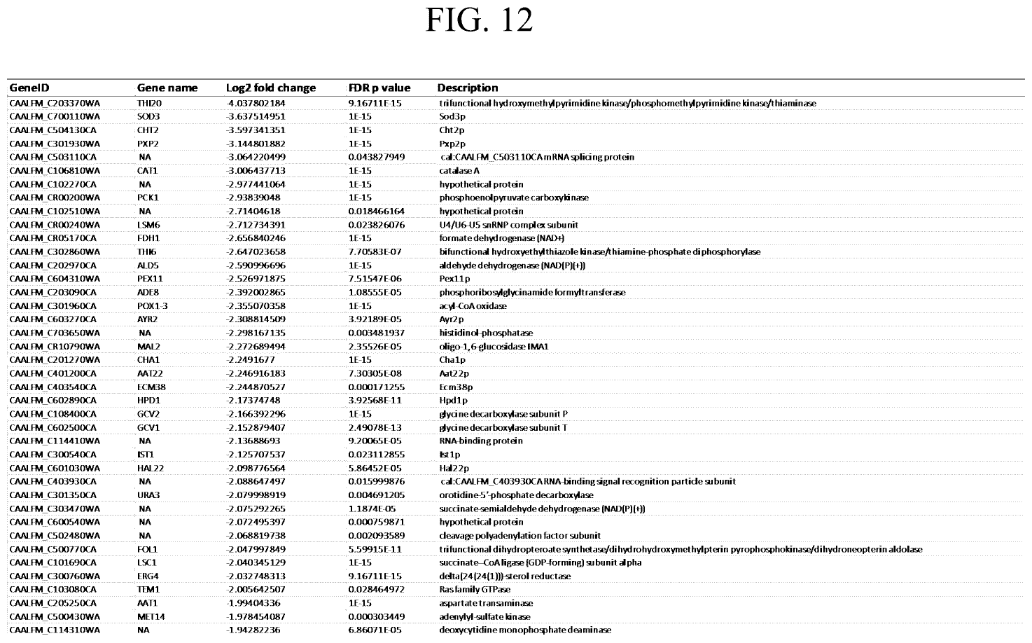

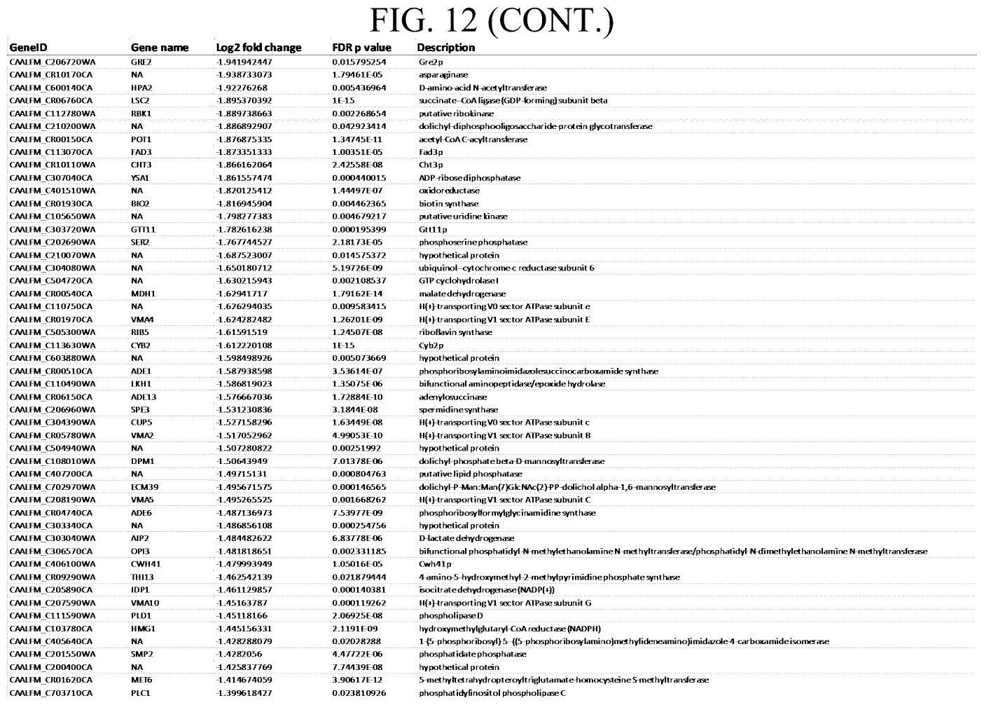

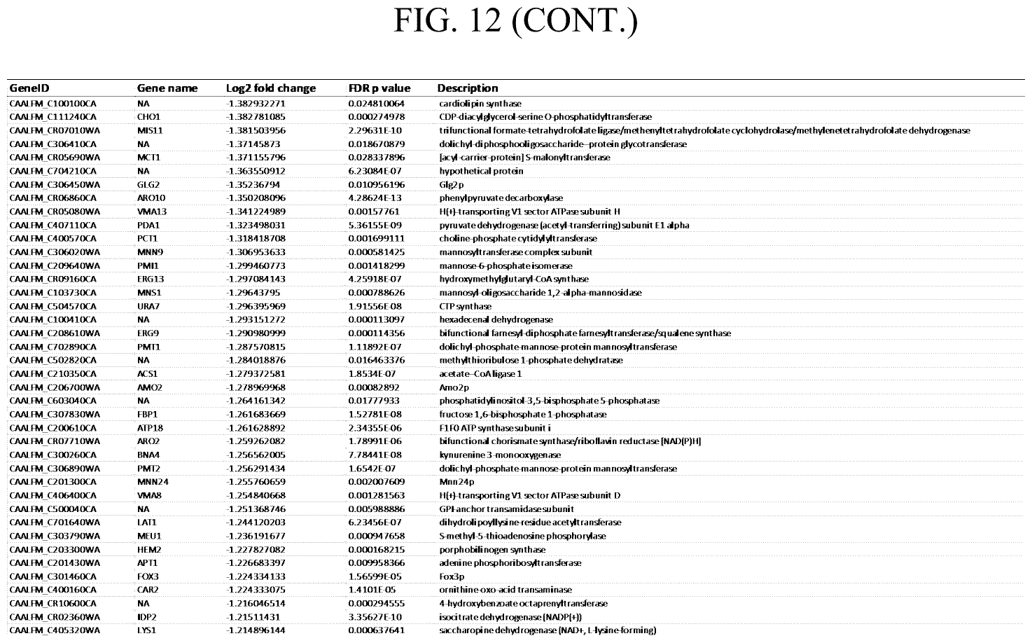

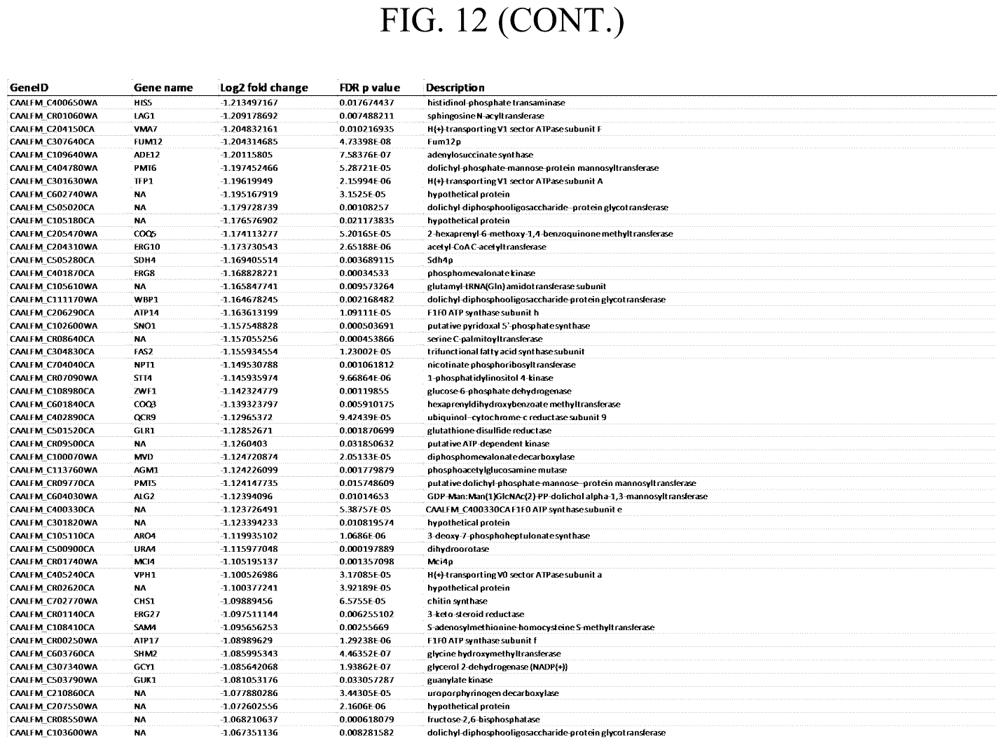

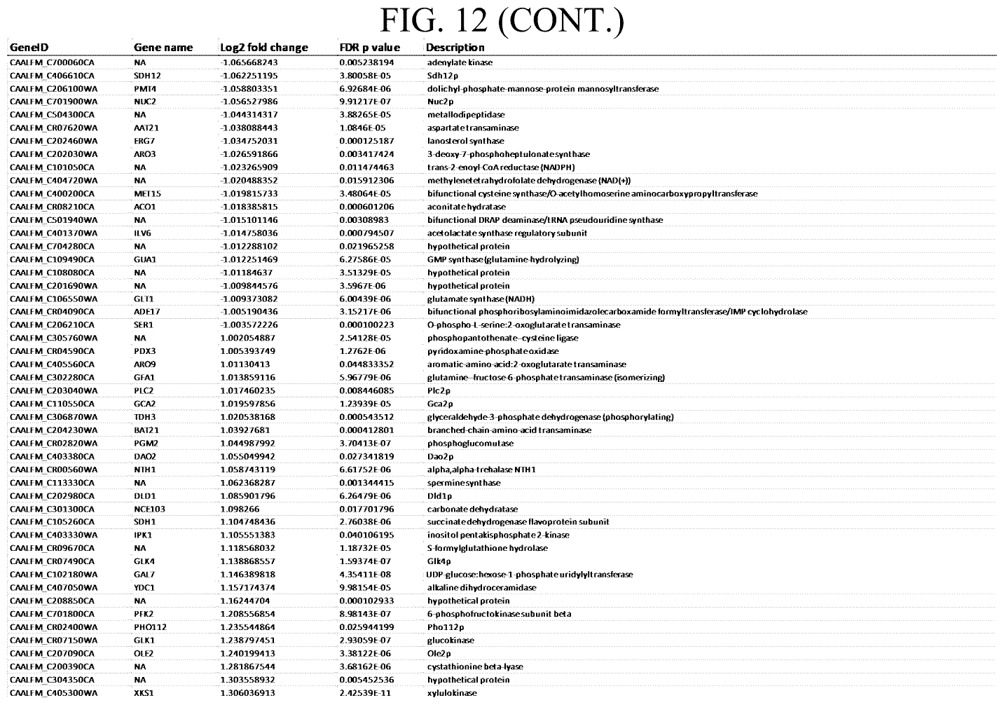

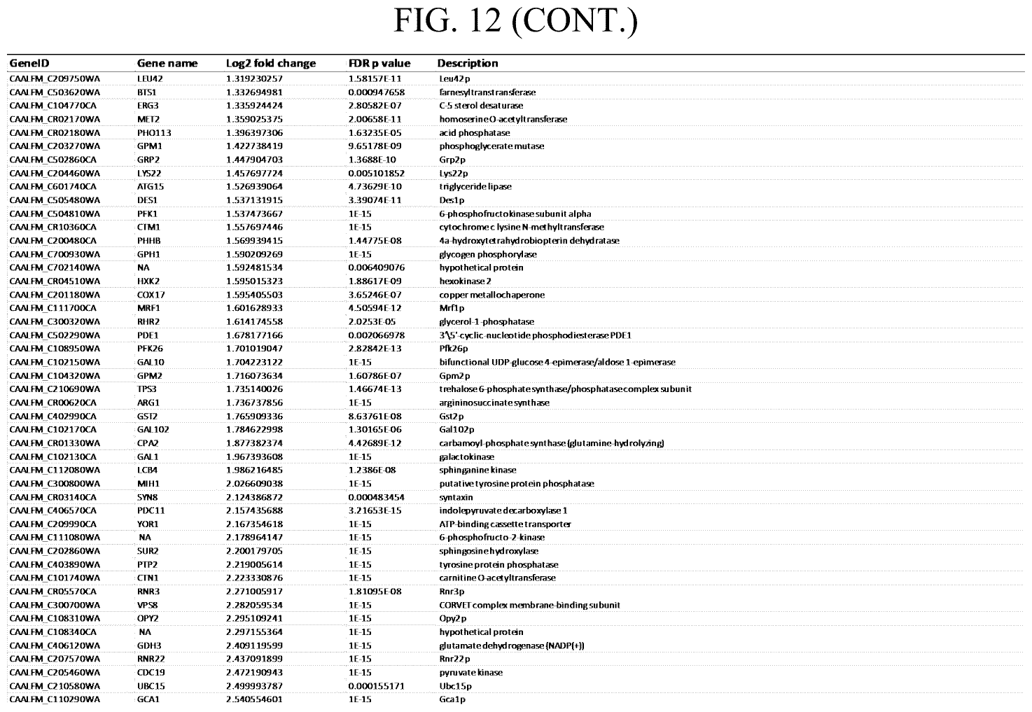

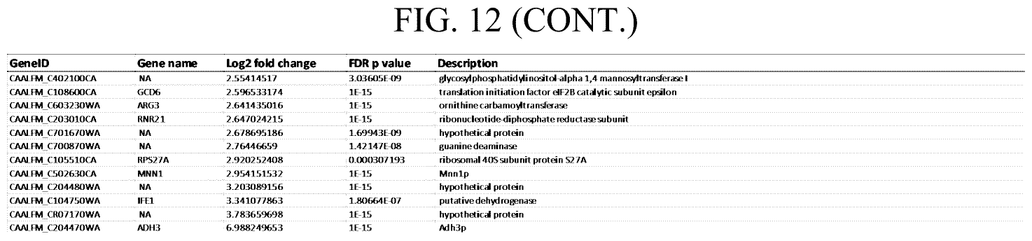

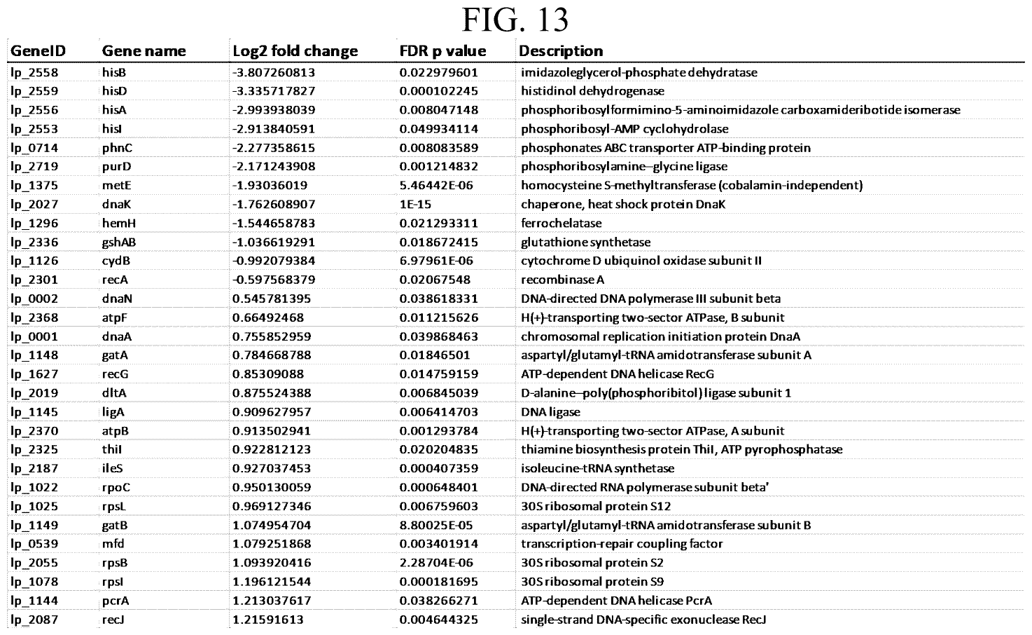

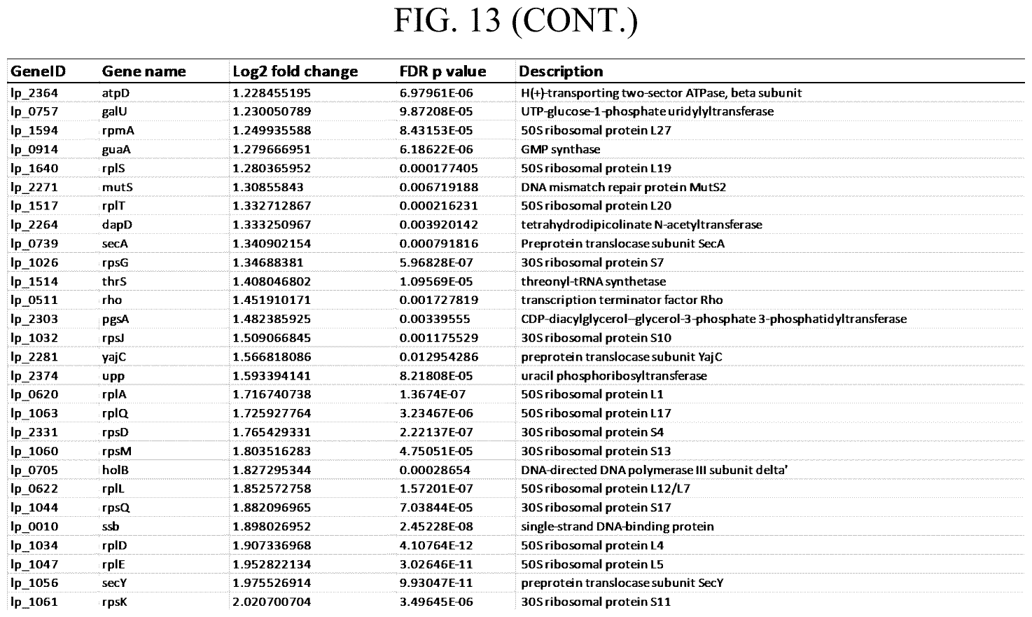

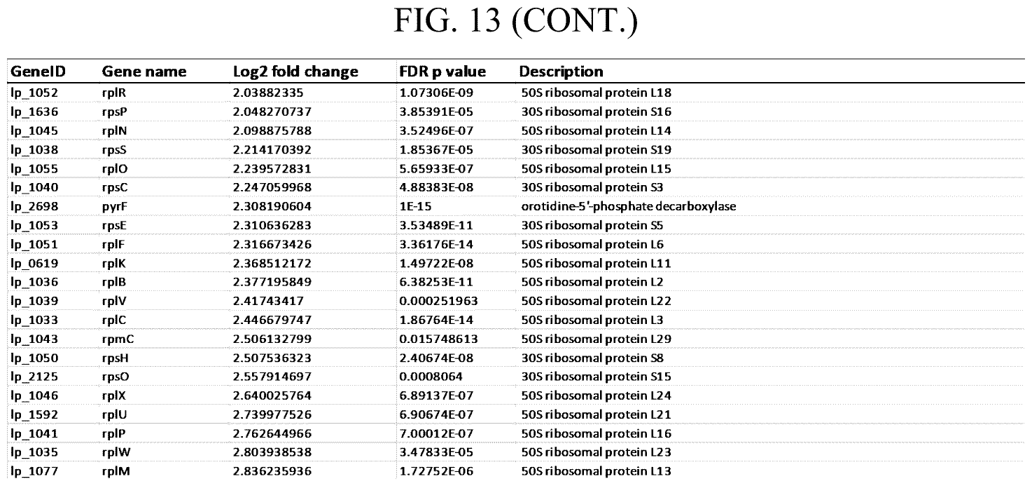

The Principal Component Analysis (PCA) and the hierarchical clustering analysis indicated distinctive transcriptomic profiles of biofilms treated by L. plantarum 14917. Overall, 441 genes of S. mutans and 232 genes of C. albicans had differential expression between L. plantarum 14917 treated multi-species biofilm and the control group; while 391 genes of L. plantarum 14917 differentially expressed between the multi-species group and L. plantarum 14917 single-species biofilms (FIGS. 11-13). Worth noting, genes related to C. albicans resistance to antifungal medication (ERG4), fungal cell wall chitin remodeling (CHT2), and resistance to oxidative stress (CAT1) were significantly downregulated when treated by L. plantarum 14917.

KEGG pathway analyses were further performed with 441 S. mutans DEGs, 232 C. albicans DEGs and 391 L. plantarum 14917 DEGs, resulting in 33 pathways for S. mutans, 66 pathways for C. albicans, and 31 pathways for L. plantarum 14917. Transcriptomic analysis revealed the disruption of S. mutans and C. albicans cross-kingdom interactions with added L. plantarum. Genes of S. mutans and C. albicans involved in metabolic pathways (e.g., EPS formation, carbohydrate metabolism, glycan biosynthesis and metabolism) were significantly downregulated. In contrast, genes of L. plantarum 14917 in the pathways of genetic information processing, environmental information processing, cellular processes, and metabolism (lipid, carbohydrate, glycan, energy) were significantly upregulated.

To determine the transcriptomic dynamic changes in genes of interest during specific stages of biofilms formation, particularly with the significant drop of pH value in the culture media, qRT-PCR were performed for biofilms at 50 h and 52 h (2 and 4 hours after culture medium change). S. mutans genes related to EPS formation (gtfB and gtfC) were significantly down-regulated at 50 h. Genes related to C. albicans resistance fungal cell wall chitin remodeling (CHT2), and resistance to oxidative stress (CAT1) were also significantly downregulated following culture medium change. Lactobacillus genes plnD, plnG and plnN that contribute to antimicrobial peptide plantaricins were significantly upregulated.

The study results revealed antimicrobial properties of the overnight culture supernatant of L. plantarum. Furthermore, the study demonstrated the dose-dependent inhibition of L. plantarum on the growth of S. mutans and C. albicans, where a threshold (10⁸ CFU/ml) of L. plantarum is needed to demonstrate the inhibitory effect in the mix-species model that mimicked high risk for dental caries. An ecological shift of microbial community was seen in the model. Despite the inhibition of S. mutans and C. albicans by a high dose of L. plantarum (≥10⁸ CFU/ml), a low dose of L. plantarum (10⁴⁻⁶ CFU/ml) promoted the growth of S. mutans and C. albicans in 1% glucose planktonic condition, which shows the mechanistic interaction between L. plantarum and other species. Among the four tested Lactobacillus spp, L. plantarum 14917 exhibited superior inhibitory properties, whereas, L. rhamnosus, a commonly used probiotic in commercials products was not capable of inhibiting the growth of C. albicans and S. mutans in cariogenic biofilms. L. plantarum has various potential pharmaceutical usages to prevent and treat respiratory diseases, irritable bowel syndrome, depression, etc, in addition to its antifungal and antibiofilm activities observed in this study. Without being bound by theory, mechanisms of action may relate to: production of plantaricins; altered fitness and virulence of S. mutans with the addition of L. plantarum 14917; altered C. albicans virulence; production of other antimicrobial product such as hydrogen peroxide and lactic acid; and sugar metabolism.

### Example 7. Effect of Probiotic L. plantarum on S. mutans and C. albicans Clinical Isolates from Children with Early Childhood Caries

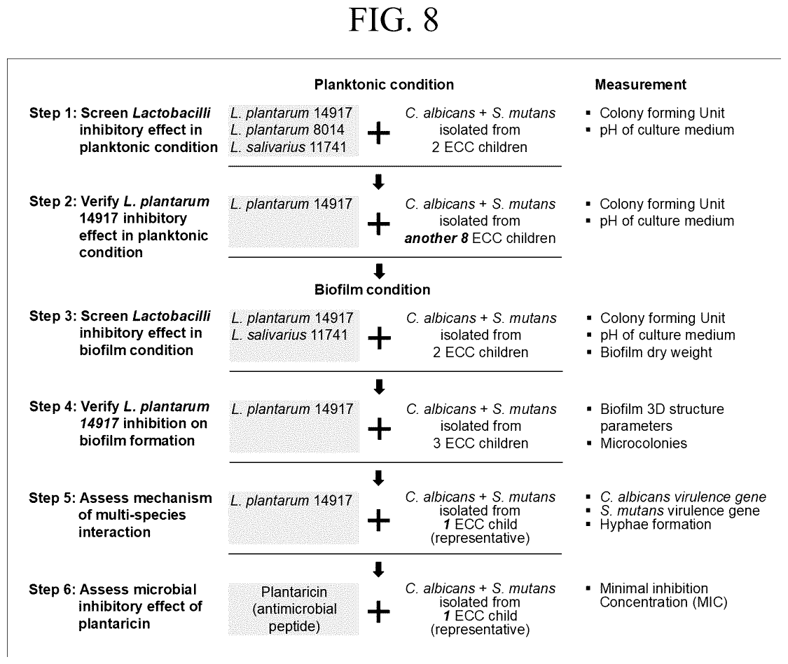

The study was designed in six steps to screen the best-performed probiotic Lactobacillus spp. on S. mutans and C. albicans clinical isolates. The study scheme is shown in FIG. 8. In Step 1, the inhibitory effect of Lactobacilli was assessed in the planktonic condition against the C. albicans and S. mutans from two S-ECC children. The best-performed Lactobacillus advanced to Step 2 to verify its inhibitory effect against C. albicans and S. mutans from additional eight S-ECC children. In Step 3, two of the three Lactobacilli with a higher inhibition on S. mutans and C. albicans in the planktonic condition were further tested with C. albicans and S. mutans isolated from two S-ECC children in a multi-species biofilm condition. In Step 4, the better-performed Lactobacillus advanced to Step 5 to assess its effect on cariogenic biofilm structure. Moreover, molecular assays were used to assess the mechanistic interactions between Lactobacillus, S. mutans, and C. albicans in biofilms in Step 5. Lastly, the effect of plantaricin, antimicrobial peptides produced by L. plantarum, on the growth of S. mutans and C. albicans was examined in Step 6.

### Example 8: Characteristics of S-ECC Children Whose S. mutans and C. albicans were Isolated

The demographic-socioeconomic-oral health condition of the S-ECC children the C. albicans and S. mutans isolated from is shown in Table 1. The S-ECC children were 3.5±1.0 years of age, with equal number of males and females. Majority of the children brushed their teeth daily, and did not attend daycare. The plaque index was 1.8±0.6. The average decayed teeth number and decayed surface number were 11.7±5.1 and 27.2±17.4, respectively.

TABLE 1
Demographics data and characteristics
of study participants (n = 10).

| Items | Mean (SD) or % |
|---|---|
| Age (Year) | 3.5 ± 1.0 |
| Gender (Male) | 50% |
| Race: Caucasian | 60% |
| African American | 30% |
| Asian | 10% |
| Ethnicity: Hispanic | 10% |
| Brushing frequency (Daily) | 90% |
| Attending daycare (Yes) | 20% |
| Plaque Index | 1.8 ± 0.6 |
| dt | 11.7 ± 5.1 |
| mt | 0.2 ± 0.6 |
| ft | 0.1 ± 0.3 |
| dmft | 12 ± 4.9 |
| ds | 27.2 ± 17.4 |
| ms | 1.0 ± 3.2 |
| fs | 0.1 ± 0.3 |
| dmfs | 28.3 ± 16.5 |

Note:
Clinical isolates were obtained from the children with ECC.

### Example 9: L. plantarum 14917 Inhibited the Growth of S. mutans and C. albicans Clinical Isolates in Planktonic Condition

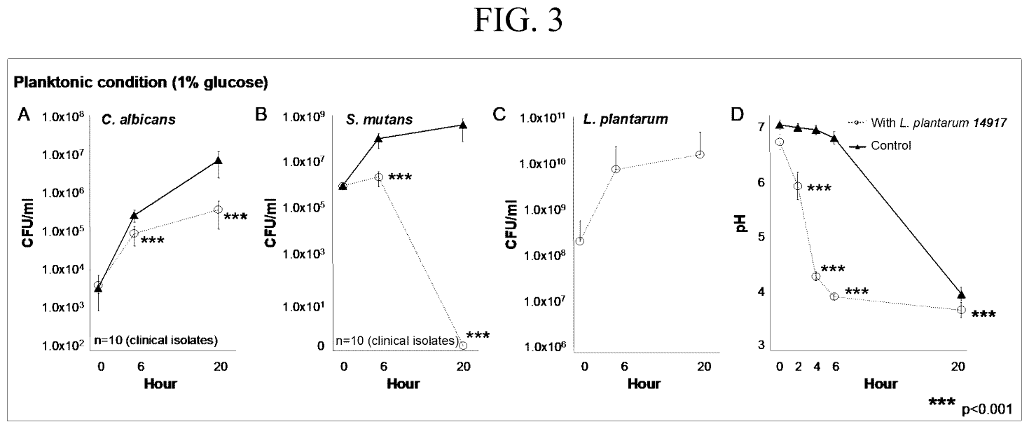

The growth of C. albicans, S. mutans, and L. plantarum 14917 in multispecies planktonic conditions are plotted in FIG. 3. C. albicans and S. mutans clinical strains were isolated from 10 children with early childhood caries (ECC). Experiments repeated in triplicates. Each planktonic multispecies condition included the C. albicans and S. mutans isolated from the same ECC child, with added L. plantarum 14917. The control group only consisted of C. albicans and S. mutans. L. plantarum inhibited the growth of C. albicans by <1 log after 6 hours and approximately 2 logs at 20 hours (p<0.05 at 20 h). L. plantarum significantly inhibited the growth of S. mutans at 6 hours by 1 log, and completely inhibited the growth of S. mutans after 20 hours (p<0.05 at 20 h). L. plantarum maintained a stable growth during the 20 hours of interaction with S. mutans and C. albicans. The culture medium pH dropped rapidly with the addition of L. plantarum in planktonic condition, and both groups reached the same acidic level at 20 hours.

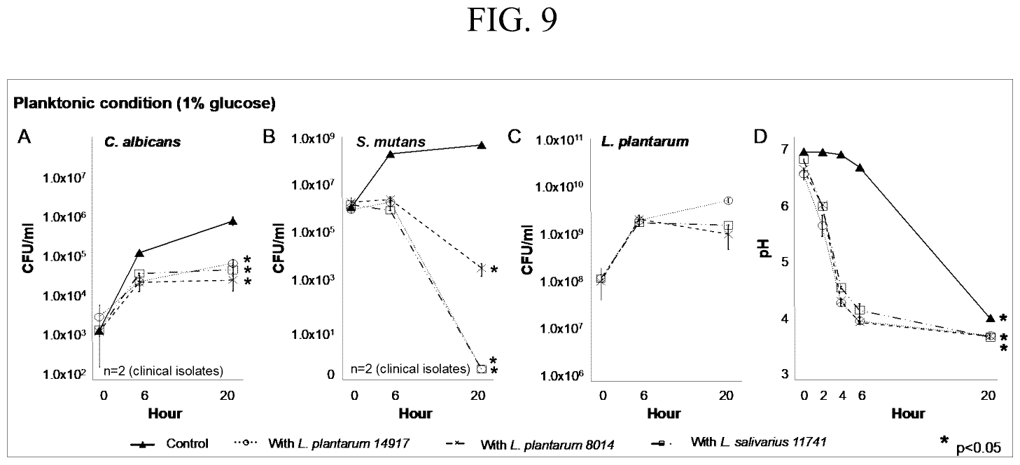

Worth noting, among the three Lactobacillus spp. tested in the screening step against C. albicans and S. mutans isolated from two S-ECC children, the inhibitory effect of L. plantarum 14917 and L. salivarius was similar, but more superior than L. plantarum 8014 (FIG. 9).

### Example 10: L. plantarum 14917 Inhibited Biofilm Formation by S. mutans and C. albicans Clinical Isolates

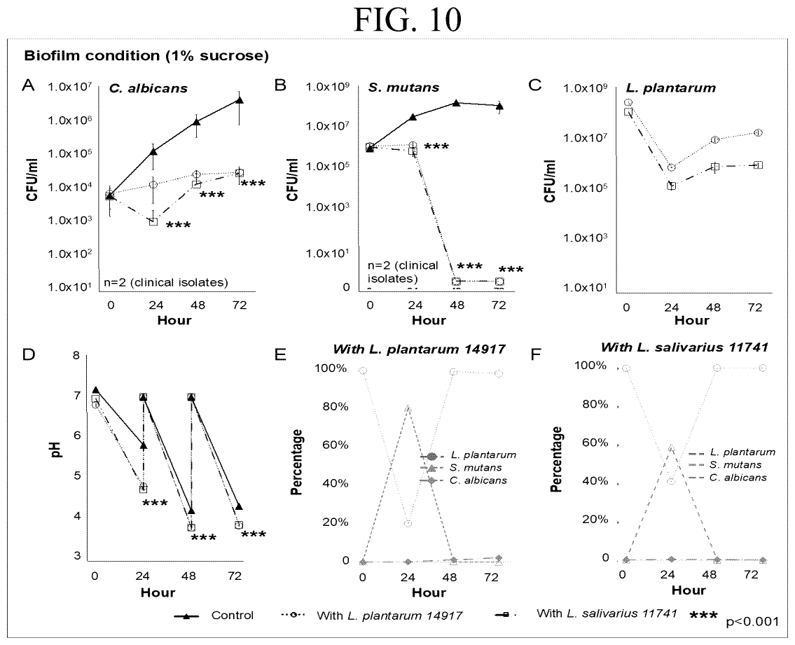

During the screening step, L. plantarum 14917 and L. salivarius 11741 were added to the biofilms formed by S. mutans and C. albicans isolated from two S-ECC children respectively. Both L. plantarum 14917 and L. salivarius 11741 inhibited the growth of C. albicans and S. mutans (FIG. 10). Although both L. plantarum 14917 and L. salivarius 11741 became the dominate species after 48 h incubation, L. plantarum 14917 had higher composition at 24 hours (40%, FIG. 10, panel E) compared to L. salivarius 11741 (20%, FIG. 10, panel F). Furthermore, the growth of L. plantarum 14917 within the multispecies biofilms remained at a higher level than L. salivarius 11741 by 2 log at 72 hours (FIG. 10, panel C). For the reasons stated above, L. plantarum 14917 advanced to the assessment of impact on biofilm structure and mechanistic interaction assessment.

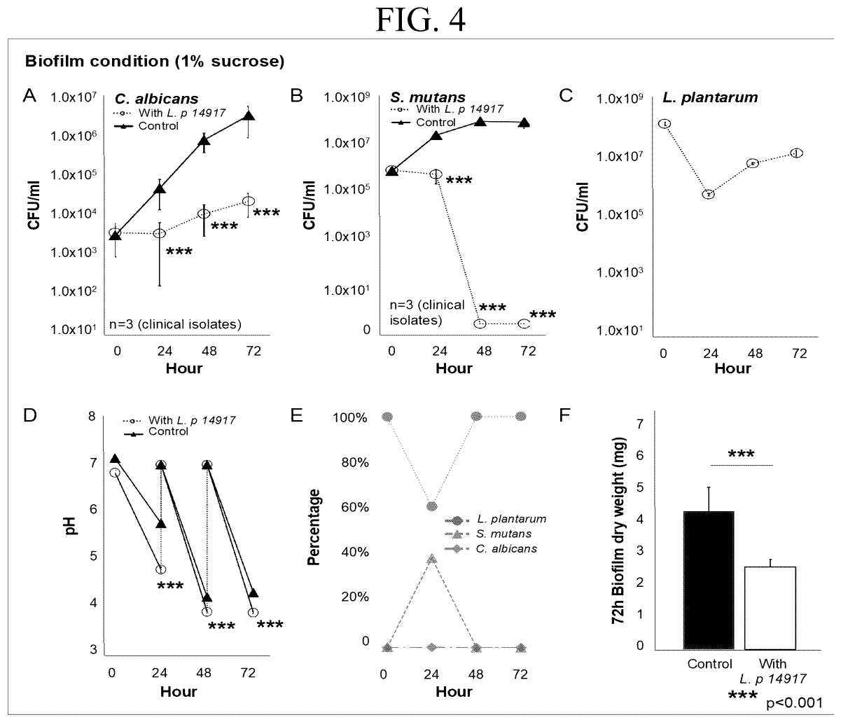

The growth of C. albicans and S. mutans were significantly inhibited by L. plantarum 14917, and is plotted in FIG. 4. In the treated group with L. plantarum 14917, C. albicans was reduced by 3 logs compared to the control group. At 48-h, after two times administration of L. plantarum 14917, S. mutans was inhibited entirely in biofilms of treatment group. L. plantarum dropped during the first 24 h due to the fact of low-sucrose (0.1%) condition and remained growing steady after 24-h within the high-sucrose (1%) condition. The pH of the culture medium was significantly lower with added L. plantarum 14917 at 24, 48, and 72-h, compared to the control group (p<0.05). L. plantarum 14917 became the dominant species after 48 hours of incubation.

### Example 11: L. plantarum 14917 Altered 3D Structure of Biofilms Formed by S. mutans and C. albicans Clinical Isolates

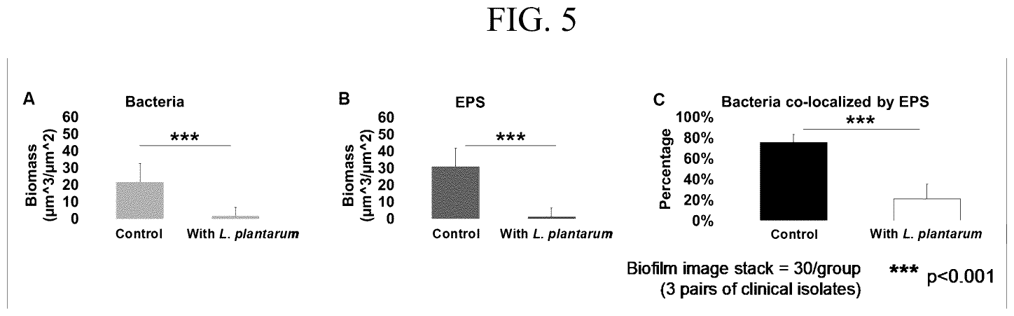

Since L. plantarum 14917 demonstrated a better inhibition of C. albicans and S. mutans isolates in planktonic and biofilm conditions, it advanced to assessing the impact on biofilm structure and mechanistic interaction assessment. L. plantarum 14917 significantly reduced cariogenic biofilm formation measured by bacteria and EPS biomass, compared to the control group (C. albicans-S. mutans duo-species biofilm). In contrast to the complex and thick biofilms formed in the control group, the L. plantarum 14917-treated biofilms significantly reduced biofilm thickness and biomass of both bacteria and EPS (FIG. 5, panels A and B, p<0.05). The horizontal coverage of the control group was also much broader than the treatment group, with nearly 60% bacterial coverage and 40% EPS coverage in the most abundant layer (˜30-40 um above the substrate), while the treated group only had 20% coverage at the most abundant layer (˜10 μm above the substrate). In addition, bacteria co-localized by EPS was significantly less in the treatment group (p<0.05).

Furthermore, the added L. plantarum 14917 also significantly impacted the microcolony formation in the 72 h multispecies biofilms. In contrast to the well-formed mushroom-shaped microcolonies in the control group, L. plantarum 14917-treated biofilms had significantly compromised microcolony structure. Microcolonies are considered virulent and functional structures of biofilm. Surface-attached and free-floating microcolonies were quantified and their numbers and size were compared between the control and L. plantarum 14917-treated biofilms. Numerous and large microcolonies were detected in control group, while the intervention of L. plantarum 14917 resulted in less and smaller size of microcolonies (Table 2).

TABLE 2
Quantitative assessment of microcolonies in multispecies biofilms

| | | With L. plantarum |
|---|---|---|
| | Control | 14917 |
| Microcolonies Parameters | (n = 30) | (n = 30) |
| Number of attached microcolonies | 15.3 ± 4.1 | 9.2 ± 6.7** |
| Area of attached microcolonies (um²) | 972.2 ± 924.4 | 283.6 ± 84.3** |
| Volume of attached microcolonies (um³) × 10³ | 3471.5 ± 2334.8 | 25.6 ± 27.5*** |
| Number of free microcolonies | 287.4 ± 71.1 | 213.3 ± 77.5*** |
| Diameter of free microcolonies (um) | 38.0 ± 4.3 | 28.4 ± 3.0*** |
| Volume of free microcolonies (um³) × 10³ | 925.0 ± 255.0 | 145.8 ± 111.8*** |

*p < 0.05,
**p < 0.01,
***p < 0.001 when comparing the control and L. plantarum 14917-treated biofilms.
Note:
n = 30 is defined that 30 biofilm image stacks were used for each group (control and treatment)

### Example 12: Plantaricin Inhibited the Growth of S. mutans and C. albicans Clinical Isolates

S. mutans (3.6×10³ CFU/ml) and C. albicans (3.1×10¹ CFU/ml) were treated by the plantaricin ranging from 0-400 ng/ml respectively, and grew for 24 h in 1% glucose condition. The MIC of plantaricin was 400 ng/ml for S. mutans and 200 ng/ml for C. albicans. The clear culture was plated and incubated for additional 48 hours. Results revealed that the inhibitory effect of S. mutans and C. albicans were bacteriostatic and fungistatic.

### Example 13: L. plantarum 14917 Downregulated C. albicans and S. mutans Virulence Genes in Biofilms

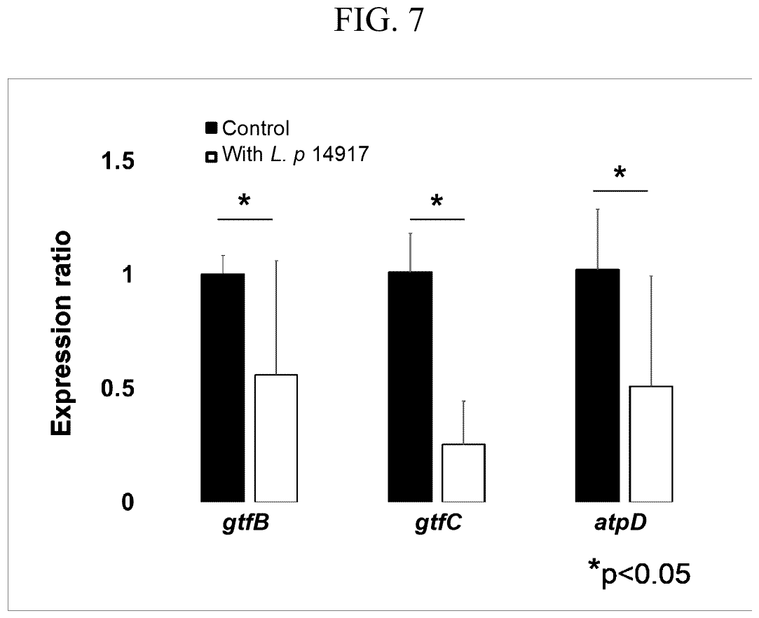

The expression of S. mutans genes related to carcinogenicity (gtfB, gtfC, and atpD) were reduced by approximately 50% in the 72 h-biofilms treated by L. plantarum 14917, comparing to the control group (see FIG. 7).

### Example 14: Inhibition on C. albicans Hyphae Formation by L. plantarum

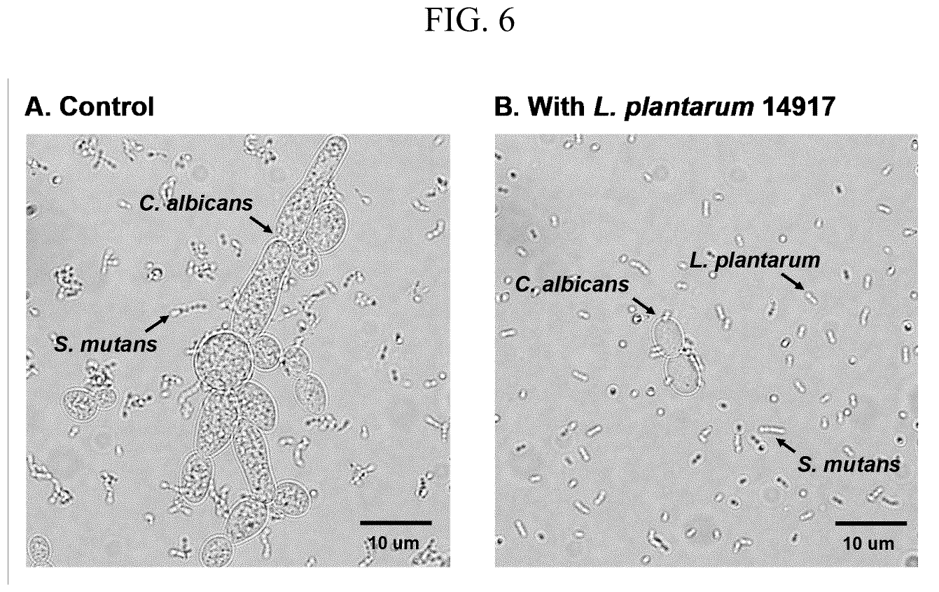

The inhibition of C. albicans switching from yeast to hyphal form was observed in the planktonic condition when treated by L. plantarum 14917. (FIG. 6)

L. plantarum 14917 demonstrated equal effectiveness in inhibiting clinically isolated C. albicans and S. mutans from S-ECC children, compared to wild-type strains, indicating L. plantarum 14917's strong potential to be incorporated into a future clinical regimen of caries prevention and control from targeting cariogenic pathogens.

The study finding showed a remarkable inhibitory effect of L. plantarum 14917 on S. mutans and C. albicans clinical isolates, resulting in a reduced biofilm structure with significantly less microbial and extracellular matrix and less-virulent microcolonies structure. The mechanistic assessment indicated that L. plantarum 14917 had a positive inhibitory impact on the expression of S. mutans and C. albicans virulence genes and virulent structure, such as C. albicans hyphae formation. Future utilization of L. plantarum 14917 and/or its antimicrobial peptide plantaricin can lead to a new paradigm shift in dental caries prevention.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A method for decolonizing or inhibiting formation of bacteria-based biofilms in a subject, comprising administering to the subject an effective amount of
(1) one or more Lactobacilli, and/or
(2) a plantaricin, wherein the one or more Lactobacillus are selected from the group consisting of L. rhamnosus ATCC 2836, L. plantarum ATCC 8014, L. plantarum ATCC 14917, and L. salivarius ATCC 11741.

2. The method of claim 1, wherein the bacteria-based biofilm is formed on tooth surfaces.

3. The method of claim 1, wherein the pharmaceutical composition is administered orally.

4. The method of claim 1, wherein the bacteria-based biofilms comprise S. mutans and/or C. albicans.

5. The method of claim 1, wherein the one or more Lactobacilli comprise L. plantarum ATCC 14917.

6. The method of claim 1, further comprising:
administering to the subject an effective amount of one or more additional agents that inhibit formation of a biofilm.

7. The method of claim 6, wherein the one or more additional agents that inhibit formation of a biofilm comprise antifungal agents.

8. The method of claim 6, wherein the one or more additional agents that inhibit formation of a biofilm comprise nystatin.

9. A method for preventing or treating a biofilm-related disease in a subject, comprising administering to the subject an effective amount of
(1) one or more Lactobacillus; and/or
(2) a plantaricin, wherein the one or more Lactobacillus are selected from the group consisting of L. rhamnosus ATCC 2836, L. plantarum ATCC 8014, L. plantarum ATCC 14917, and L. salivarius ATCC 11741.

10. The method of claim 9, wherein the biofilm-related disease is selected from the group consisting of dental caries, oral yeast infections, periodontitis, peri-implantitis, and burning mouth syndrome.

11. The method of claim 10, wherein the dental caries is early childhood caries (ECC).

12. The method of claim 9, wherein the biofilm-related disease is caused by bacteria-based biofilms that comprise S. mutans and/or C. albicans.

13. The method of claim 9, wherein the one or more Lactobacilli comprise L. plantarum ATCC 14917.

14. The method of claim 9, further comprising:
administering to the subject an effective amount of one or more additional agents that inhibit formation of a biofilm.

15. The method of claim 14, wherein the one or more additional agents that inhibit formation of a biofilm is an antifungal agent.

16. A method for preventing development of early childhood caries (ECC) in a newborn subject, comprising:
administering to the mother of the newborn subject during pregnancy, an effective amount of a pharmaceutical composition comprising one or more Lactobacilli selected from the group consisting of L. rhamnosus ATCC 2836, L. plantarum ATCC 8014, L. plantarum ATCC 14917, and Z. salivarius ATCC 11741.

17. A pharmaceutical composition, comprising:
one or more Lactobacillus selected from the group consisting of Lactobacillus rhamnosus ATCC 2836, Lactobacillus plantarum ATCC 8014, Lactobacillus plantarum ATCC 14917, and Lactobacillus salivarius ATCC 11741; and
a pharmaceutically acceptable carrier, wherein the pharmaceutical composition is formulated as a mouthwash, dental gel or dental coating.

18. The pharmaceutical composition of claim 17, wherein the pharmaceutical composition has a pH value in the range of 4-7.

19. The pharmaceutical composition of claim 17, wherein the pharmaceutical composition further comprises 1% sugar (w/v).

20. The pharmaceutical composition of claim 17, comprising L. plantarum ATCC 8014 and L. plantarum ATCC 14917 at a L. plantarum ATCC 8014:L. plantarum ATCC 14917 CFU ratio in the range of 1:10 and 10:1.
